(19) United States
(12) Patent Application Publication
(10) Pub. No.: US 2025/0099434 A1
(43) Pub. Date: Mar. 27, 2025

(54) METHODS OF TREATING AMYLOID RELATED BRAIN DISORDERS USING NOVEL COMPOUNDS AND ANTIBODIES

(71) Applicant: T3D Therapeutics, Inc., Research Triangle Park, NC (US)

(72) Inventors: Stanley D. CHAMBERLAIN, Wilson, WY (US); John DIDSBURY, Sunset Beach, NC (US); Warren J. STRITTMATTER, Asheville, NC (US)

(21) Appl. No.: 18/833,293

(22) PCT Filed: Jan. 23, 2023
(86) PCT No.: PCT/US2023/011332

§ 371 Date: Jul. 25, 2024

Related U.S. Application Data
(60) Provisional application No. 63/303,176, filed on Jan. 26, 2022.

(51) Int. Cl. A61K 31/421; A61K 39/00; A61P 25/28; C07K 16/18

(52) U.S. Cl. CPC A61K 31/421; A61P 25/28; C07K 16/18; A61K 2039/505

(57) ABSTRACT

Novel indane acetic acid compounds alone or in combination with anti-amyloid beta antibodies, for the treatment of Alzheimer's disease, for the reduction of Amyloid-related imaging abnormalities (ARIA), and for the treatment of Cerebral Amyloid Angiopathy and vasogenic edema (VE).

### CROSS-REFERENCE TO RELATED APPLICATIONS

The present application claims priority to U.S. Provisional Application Ser. No. 63/303,176 filed on Jan. 26, 2022, the contents of which are incorporated by reference herein in its entirety.

### FIELD

The present disclosure relates to the use of indane acetic acids and their derivatives, which are dual PPAR delta and gamma agonists, alone or in combination with Amyloid Beta directed antibodies, for the treatment of Alzheimer's disease, for the reduction of Amyloid-related imaging abnormalities (ARIA), and for the treatment of Cerebral Amyloid Angiopathy and vasogenic edema (VE).

### BACKGROUND

Indane acetic acids PPAR agonists detailed herein were previously disclosed in Lowe et al. US 2005/0075338 A1 Apr. 7, 2005, which is Continuation of application Ser. No. 10/205,839, filed on Jul. 25, 2002, now (U.S. Pat. No. 6,828,335 B2; 2004). The Lowe patent (U.S. Pat. No. 6,828,335 B2; 2004) is a composition of matter patent. The instant disclosure is directed to methods of use for the compounds of the indane acetic acids described in U.S. Pat. No. 6,828,335 B2; 2004.

PPARs (peroxisome proliferator-activated receptors, are a family of ligand-activated transcription factors that play an essential role in cellular processes such as cell differentiation, inflammation, and metabolism. The PPARs are ligand-activated transcription factors belonging to the nuclear hormone receptor superfamily, and exist as three different isoforms: PPARα, PPARδ (also called β), and PPARγ. PPARs are activated by lipids and fatty acid derivatives, and they carry out essential functions in lipid homeostasis, glucose metabolism, energy production and cellular differentiation. PPARs are expressed in a variety of CNS related cell types including microglia, astrocytes, oligodendrocytes and neurons. PPAR activation can modulate the immune response, stimulate metabolic and mitochondrial function, promote axon growth, and induce formation of myelinating oligodendrocytes among other disease modifying affects. Compounds which have individual, or single, PPARα, PPARδ, or PPARγ agonist activity are thought to have potential as systemic therapeutics. Dual or triple PPAR isoform agonists are not well studied and their potential as therapeutics is not well understood. Only recently have there been discovered compositions with dual PPARδ and PPARγ agonist activity where PPARδ activity is greater than PPARγ and PPARα activity.

For a therapeutic to be effective in treating a neurodegenerative disease, it must be able pass from blood through the blood-brain barrier (BBB) and into the brain extracellular fluid (BECF) in the central nervous system (CNS). The blood-brain barrier is formed by endothelial cells, which are connected by tight cell junctions. The blood-brain barrier allows the passage of water, gases, and lipid-soluble molecules by passive diffusion, as well as the selective transport of molecules such as glucose and amino acids that are crucial to neural function. The blood-brain barrier also eliminates lipophilic molecules by way of an active transport mechanism mediated by P-glycoprotein (P-gp), or other efflux transporters such as Organic anion transporter 3 (Oat3) and the peptide transporter 2 (PEPT2). For a neurodegenerative therapeutic to be effective, it has to achieve balance between passive diffusion in through the BBB, and active elimination out of the brain by the P-gp transporter, or other transporters. P-gp is an ATP-dependent, drug efflux pump for xenobiotic compounds with broad tissue distribution including the endothelia cells of the BBB. One measure of whether a small molecule penetrates the BBB and is not rapidly transported out, is the brain to plasma ratio of the drug. This is measured in pre-clinical animal models by determining plasma concentration vs time curves as in a standard pharmacokinetic study, and in addition, harvesting brains and determining whole brain concentrations over time. The brain to plasma concentration ratio can then be determined at any time point, such as by the Cₘₐₓ, or for the entire time curve (AUC, area under the curve). Alternatively, brain exposure can be determined by measuring drug concentrations in ventricular and lumbar cerebrospinal fluid (CSF). Clinically, brain FDG-PET can be used as an indirect measure of the pharmacological effect of a therapeutic and of brain levels of drug.

The brain has many different cell types. PPARs are known to be involved in the biology of the different brain cells.

Microglia are the primary immune effector cells of the central nervous system (CNS). Neurodegenerative diseases often involve inflammation. Activated microglia release inflammatory cytokines that further recruit circulatory monocytes to the injured brain tissue. PPARγ and PPARδ are critical transcriptional modulators that influence microglia phenotype. PPAR activation of microglia promotes phagocytosis of protein aggregates and is neuroprotection. Thus, PPAR induced microglia might be beneficial by promoting the removal of cellular debris such as amyloid beta oligomers or plaques.

Astrocytes are the most numerous and diverse non-neuronal support cells (neuroglial) in the CNS, and display a remarkable heterogeneity in their morphology and function. They have numerous projections that anchor neurons to their blood supply. Astrocytes can release damaging inflammatory molecules that cause neuron loss. Modulating inflammation is one of the best-studied roles of PPAR activation in astrocytes and has been examined in multiple experimental models. The responsiveness of astrocytes to PPAR agonists, may position astrocytes as an important role in treatment of neurodegenerative disease.

Oligodendrocytes are myelinating cells of the CNS, which are vulnerable to both inflammatory cytokines and chemokines, and to oxidative damage from reactive oxygen species (ROI). Loss of myelinating oligodendrocytes exposes axons to cytokines and ROIs, leading to axon degeneration. Since myelin is composed of lipid, and PPARs play a major role in lipid metabolism, PPARs may help regulate the function of oligodendrocytes. Targeting oligodendrocytes through PPAR agonists may enhance production and maturation of oligodendrocyte precursor cells (OPCs) and repopulate lost oligodendrocytes and maintain myelination and the integrity of axons.

Neuron loss is a devastating and permanent effect of neurodegeneration. Mitochondrial dysfunction correlates with neuronal cell death, functional impairment, and cognitive deficit since energy production by mitochondria is essential for survival of all cells including neurons. PPAR agonists have been extensively studied for their role in modulating metabolism and energy production. Activation of PPAR receptors by fatty acids promotes mitochondrial β-oxidation increasing cellular energy production. PPAR agonists may thus induce a clearance mechanism for the amyloid-beta.

Alzheimers Disease (AD) a chronic, progressive, neurodegenerative disease that is the cause of 60% to 70% of cases of dementia. The most common early symptom is short-term memory loss, and as the disease advances, symptoms can include language difficulties, disorientation, mood swings, loss of motivation, poor self care, and behavior issues. Over time, bodily functions are lost, ultimately leading to death. The average life expectancy following diagnosis is three to nine years. The cause of Alzheimer's disease is not well understood. About 70% of the risk is believed to be genetic. Type 2 Diabetes is also a significant risk factor, along with a history of head injuries, depression, or hypertension. The disease process is associated with amyloid plaques and tau neurofiburilary tangles in the brain. Thus, in one aspect of the present disclosure, Alzheimers Disease is an amyloid-related brain disorder. Diagnosis is based on a history of the illness and cognitive testing with medical imaging. Postmortem examination of brain tissue is needed for a definite diagnosis. Alzheimer's disease is characterized by loss of neurons and synapses in the cerebral cortex and certain subcortical regions. This loss results in gross atrophy of the affected regions, including degeneration in the temporal lobe and parietal lobe, and parts of the frontal cortex and cingulate gyrus. Studies using MRI and PET have documented reductions in the size of specific brain regions in people with AD as they progressed from mild cognitive impairment to Alzheimer's disease. Many different brain cell types such as astrocytes, microglia, oligodendrocytes and white matter and gray matter neurons are thought to play a role in AD neurodegeneration. There are approximately 30 million people worldwide with AD, mostly in people over 65 years of age, and leads to about 2 million deaths per year. There are no medications to stop or reverse the progression of AD.

Amyloid-related imaging abnormalities (ARIA) are abnormal differences seen in magnetic resonance imaging (MRI) of the brain of Alzheimer's disease patients, associated with amyloid-modifying therapies, particularly human monoclonal antibodies such as aducanumab. Thus, in one aspect of the present disclosure, an amyloid-related brain disorder comprises the presence of an amyloid-related imaging abnormality (ARIA). There are two types of ARIA: vasogenic edema and sulcal effusions (ARIA-E) and hemosiderin deposits (ARIA-H).

ARIA-E or extracellular brain edema, also called vasogenic edema, is an aberrant increase in the permeability of the blood-brain barrier. The breakdown of the normally tight endothelial junctions that make up the blood-brain barrier allows extravasation of fluid, ions, and plasma proteins into the brain which is detected by MRI. In one aspect of the present disclosure, vasogenic edema is an amyloid-related vasogenic edema. In another aspect of the disclosure, general vasogenic edema that is not the result of an amyloid-related brain disorder is contemplated as a non-amyloid vasogenic edema.

ARIA-H represents a spectrum of MRI findings due to haemosiderin deposition again resulting from breakdown of vascular integrity. Haemosiderin is an iron containing ferritin complex that forms when blood leaves a ruptured blood vessel and the iron containing hemoglobin is released into the extracellular space. Macrophages engulf the hemoglobin to degrade it, producing iron containing hemosiderin, which can be detected by MRI. It is thought that cerebral micro-hemorrhages, cerebral edemas, or vasogenic edemas, are related to amyloid burden and to the removal of amyloid plaques from cerebral blood vessels by amyloid targeted antibodies. Amyloid-related imaging abnormalities (ARIA) have been reported in AD patients treated with bapineuzumab, a humanized monoclonal antibody to amyloid-β. ARIA includes MRI signal abnormalities suggestive of ARIA-E and ARIA-H. Over the past decade since amyloid-modifying therapeutic agents have entered AD clinical trials, the occurrence of MRI abnormalities has required careful consideration by academic investigators, pharmaceutical companies and regulatory authorities. MRI signal changes, thought to represent vasogenic edema (VE) and cerebral micro-hemorrhages (mH), were first observed in trials of a monoclonal antibody against amyloid β and have since been associated with other amyloid-modifying therapies. Arrighi et al., 2016; Barakos et al. 2013; and Sperling et al., 2011, each of which is herein incorporated by reference with regard to such background teachings.

Cerebral amyloid angiopathy (CAA) is the accumulation of amyloidogenic proteins, most often amyloid β (Aβ), in cerebral blood vessel walls, leading to a weakened vasculature and thereby creating a major risk for intracerebral hemorrhages (ICH). Thus, in one aspect of the present disclosure, CAA is an amyloid-related brain disorder. Several types of hereditary disorders exist that result in CAA, caused by missense mutations within the Aβ precursor protein gene. However, CAA most frequently occurs sporadically and is observed in cognitively normal elderly, but also frequently in patients with AD. In patients with AD, CAA is tightly linked to the development of ARIA, a frequently occurring side-effect of anti-Aβ immunotherapy defined by neuroimaging (eg, ≈40% of AD patients treated with aducanumab develop ARIA). Jäkel et al., 2021, herein incorporated by reference with regard to such background teaching. Patients with CAA may present with a broad clinical spectrum, including cognitive decline, lobar ICH, and transient focal neurological episodes (recurrent, stereotyped, transient episodes of smoothly spreading paraesthesias, numbness or weakness, lasting typically seconds to minutes, usually resolving over a similar period). A rare complication of the disease is CAA-related inflammation, characterized by headache, seizures, behavioral change, focal neurological signs, impaired consciousness. CAA is a major cause of primary intracerebral hemorrhage. Cerebrovascular deposition of the ß-amyloid peptide also appears to cause ischemic brain injury. Advanced CAA is associated with various neuropathological or neuroimaging markers of non-hemorrhagic tissue injury, including microinfarcts, white matter lesions, and altered diffusion-tensor properties. These multifocal lesions likely contribute to the association between advanced CAA and cognitive impairment observed in elderly individuals. A definite diagnosis of CAA can only be obtained by post mortem neuropathological assessment of brain tissue. For the diagnosis of CAA during life, criteria have been developed, which make use of MRI to enable the diagnosis of “probable” or “possible” CAA. These “Boston criteria” are based on two CAA-related imaging markers: strictly lobar cerebral microbleeds (small brain bleeds restricted to cortical and subcortical regions of the brain), and cortical superficial siderosis (deposition of blood breakdown products in the cortical sulci over the convexity of the cerebral hemispheres). Systematic review and meta-analysis of the literature, provides reliable estimates of the prevalence of CAA pathology and MRI imaging markers of CAA in AD patients, the general elderly population, cognitively normal elderly, and in patients with lobar Intra Cerebral Hemorrhage) ICH. Almost a quarter of the elderly general population has moderate-to-severe CAA pathology. Also, in AD patients (48%) and patients with lobar ICH (57%), CAA is highly prevalent. Since CAA is associated with the development of ARIA in anti-AB immunotherapy and with a growing spectrum of clinical symptoms, there is a growing awareness of the high prevalence of CAA.

The in vivo diagnosis of cerebral amyloid angiopathy (CAA) is currently based on MRI, which largely rely on hemorrhagic features on brain magnetic resonance imaging. FDG (¹⁸F-fluoro-deoxy-D-glucose) PET (positron emission tomography), has been studied to improve in vivo diagnosis of CAA. Studies have shown that FDG uptake is reduced in posterior cortical areas, particularly the primary occipital cortex, including the including the visual cortex, which pathologically bear the brunt of vascular Aβ deposition. Significant hypometabolism (P range, 0.047 to <0.0001) has been observed in the posterior cortical areas, including the superior and inferior parietal, primary visual, lateral occipital, lateral temporal, precuneus, and posterior cingulate regions of interest, in CAA patients. This suggests that FDG PET data is relevant to the study of CAA and ARIAs.

One aspect of the present disclosure are antibodies directed to amyloid beta and are anti-amyloid beta antibodies. The present disclosure contemplates the use of any anti-amyloid beta antibody, including the specific ones described herein.

ADUHELM™ (aducanumab) is an amyloid beta-directed antibody indicated for the treatment of Alzheimer's disease sold by Biogen. Treatment with ADUHELM™ is for patients with mild cognitive impairment or mild dementia stage of disease, the population in which treatment was initiated in clinical trials. This indication is approved under accelerated approval based on reduction in amyloid beta plaques observed in patients treated with aducanumab. Titration of the aducanumab dose is required for treatment initiation. The recommended maintenance dosage is 10 mg/kg administered as an intravenous infusion over approximately one hour every four weeks. Patients obtain a recent (within one year) brain MRI prior to initiating treatment. MRIs should be obtained prior to the 7th and 12th infusions. If radiographic severe ARIA-H is observed, treatment may be continued with caution only after a clinical evaluation and a follow-up MRI demonstrates radiographic stabilization (i.e., no increase in size or number of ARIA-H). Dilution in 100 mL of 0.9% Sodium Chloride Injection, USP, is required prior to administration. Doses can be 170 mg/1.7 mL (100 mg/mL) solution in a single-dose vial or 300 mg/3 mL (100 mg/mL) solution in a single-dose vial. ADUHELM™ can cause amyloid related imaging abnormalities-edema (ARIA-E), which can be observed on MRI as brain edema or sulcal effusions, and amyloid related imaging abnormalities hemosiderin deposition (ARIA-H), which includes microhemorrhage and superficial siderosis. ARIA-E was observed in 35% of patients treated with ADUHELM™ 10 mg/kg, compared to 3% of patients on placebo. The incidence of ARIA-E was higher in apolipoprotein E ε4 (ApoE ε4) carriers than in ApoE ε4 non-carriers (42% and 20%, respectively). The majority of ARIA-E radiographic events occurred early in treatment (within the first 8 doses), although ARIA can occur at any time. Among patients treated with a planned dose of ADUHELM™ 10 mg/kg who had ARIA-E, the maximum radiographic severity was mild in 30%, moderate in 58%, and severe in 13% of patients. Resolution occurred in 68% of ARIA-E patients by 12 weeks, 91% by 20 weeks, and 98% overall after detection. 10% of all patients who received ADUHELM™ 10 mg/kg had more than one episode of ARIA-E. ARIA-H associated with the use of ADUHELM™ 10 mg/kg was observed in 21% of patients treated with ADUHELM™ 10 mg/kg, compared to 1% of patients on placebo. Clinical symptoms were present in 24% of patients treated with ADUHELM™ 10 mg/kg who had an observation of ARIA (-E and/or -H), compared to 5% of patients on placebo. The most common symptom in patients treated with ADUHELM™ 10 mg/kg with ARIA was headache (13%). Other frequent symptoms were confusion/delirium/altered mental status/disorientation (5%), dizziness/vertigo (4%), visual disturbance (2%), and nausea (2%). Serious symptoms associated with ARIA were reported in 0.3% of patients treated with ADUHELM™ 10 mg/kg. Clinical symptoms resolved in the majority of patients (88%) during the period of observation. Enhanced clinical vigilance for ARIA is recommended during the first 8 doses of treatment with ADUHELM™, particularly during titration, as this is the time the majority of ARIA was observed.

Bapineuzumab is a humanized monoclonal antibody may have potential therapeutic value for the treatment of AD. Bapineuzumab lowered key biomarkers of AD: amyloid brain plaque and hyperphosphorylated tau protein in CSF, but failed to produce significant cognitive improvements in patients in two major trials, despite lowering. Bapineuzumab is an antibody to the beta-amyloid (Aβ) plaques that are believed to underlie AD neuropathology. It binds to the extreme N-terminal 5 residues of Aβ peptide. In a previous clinical trial targeting human beta amyloid, (AN-1792), demonstrated positive outcomes with removal of plaques, but 6% but was stopped for safety reasons. Bapineuzumab was the first beta-amyloid antibody to be found to cause amyloid-related imaging abnormalities (ARIA), including an accumulation of fluid in brain tissue (ARIA-E) in patients receiving the highest dose. No health risks were found in subjects receiving either 0.5 or 1 mg of bapineuzumab. Common doses of Bapineuzumab include: doses of bapineuzumab (0.15 mg/kg, 0.5 mg/kg, 1.0 mg/kg and 2.0 mg/kg). Dosing with Bapineuzumab can be either subcutaneous or intravenous.

Gantenerumab is a fully human IgG1 antibody, which binds with sub-nanomolar affinity to a conformational epitope on Aβ fibrils. It binds to both N-terminal and central amino acids of Aβ. Gantenerumab is thought to disassemble and degrade amyloid plaques in the CNS by recruiting microglia and activating phagocytosis. Gantenerumab preferentially interacts with aggregated brain Aβ, both parenchymal and vascular. The antibody elicits phagocytosis of human Aβ deposits in AD brain slices co-cultured with human macrophages. In APP/PS-1 transgenic mice, gantenerumab binds to cerebral Aβ, reduces small plaques by recruiting microglia, and prevents new plaque formation. Gantenerumab does not alter plasma Aβ. Gantenerumab is generally safe and well-tolerated, but amyloid-related imaging abnormalities (ARIA) are observed in perhaps one third of patients at the highest dose, including both focal areas of inflammation or vasogenic edema on MRI scans in brain areas with the most amyloid reduction. Roche started a Phase 2 trial of 105 or 225 mg gantenerumab injected subcutaneously once a month into 360 participants, and in 2012 expanded the study to a Phase 2/3 registration trial of 799 people. Called Scarlet Road, this multinational, 159-center study of gantenerumab's effect on cognition and function in prodromal Alzheimer's disease delivered treatment for two years with the option of a two-year extension. The trial was stopped in December of 2014 based on an interim futility analysis. A Phase 3 trial with Gantenerumab dosed as high as 1200 mg subcutaneously. Gantenerumab lowered brain amyloid by an average of 59 centiloid on florbetapir PET, with half of the 28 participants who reached this timepoint falling below the threshold for amyloid positivity, and the rest on trajectory to do so. About one-third of participants in the extension studies developed ARIA-E. Clinical trial data from Roche shows that Gantenerumab treatment led to a large reduction in amyloid plaque as per PiB PET scans, and it normalized CSF Aβ42. Gantenerumab normalized elevated levels of CSF total tau and p-tau181, and slowed the rise of CSF neurofilament light. No clinical trial with Gantenerumab has shown a statistically significant improvement in cognition. In further analyses of the two-year open-label extension data Roche showed that ARIA tends to occurs in hotspots of rapid amyloid removal, but is not required for amyloid clearance across the brain. In current studies use monthly shots of 120 mg, ramping up to a target dose of 255 mg weekly.

Solanezumab is a humanized monoclonal IgG1 antibody directed against the mid-domain of the Aβ peptide. It recognizes soluble monomeric, not fibrillar, Aβ. The therapeutic rationale is that it may exert benefit by sequestering Aβ, shifting equilibria between different species of Aβ, and removing small soluble species of Aβ that are directly toxic to synaptic function. In Phase 2, trials administering 100 to 1,600 mg per month of solanezumab for 12 weeks, and monitoring for safety and biomarker effects for one year, confirmed the antibody's safety and tolerability. Phase 2 showed dose-dependent increases of various Aβ species in plasma and CSF but no effects on the ADAS-Cog, or indication of clinical benefit. In July 2013, Lilly started EXPEDITION-3, a 39-center Phase 3 trial in 2,100 patients with mild AD and demonstrated brain amyloid burden. In March 2016, Lilly announced that it would change the primary outcome for this trial. The trial used ADAS-Cog as a single primary and ADCS-iADL as a secondary outcome. On Nov. 23, 2016, Lilly announced, based on top-line results, that solanezumab had missed its primary endpoint in this trial. Primary and secondary outcome results were trending in the direction of a treatment benefit, but effects were small and fell short of statistical significance. Clinical biomarker data showed solanezumab treatment caused a steep increase in CSF Aβ42, indicating target engagement. Solanezumab did not alter tau biomarkers, and changed CSF neurofilament light concentrations for the worse. Changes in plasma amyloid-beta were similar to those seen in previous studies, and the differences between treatment and placebo groups were statistically significant. Changes in amyloid deposition as measured by positron emission tomography (PET) imaging did not reach statistical significance between treatment and placebo groups. The incidence of vasogenic edema (ARIA-E or amyloid-related imaging abnormality-edema/effusions) was approximately 0.1 percent of patients treated with solanezumab and 0.3 percent of patients on placebo.

Crenezumab is a passive immunotherapy approach in which patients are treated with monoclonal antibodies that specifically recognize Aβ peptides. Crenezumab recognizes multiple forms of aggregated Aβ, including oligomeric and fibrillar species and amyloid plaques with high affinity, and monomeric Aβ with low affinity. It uses a humanized antibody with an IgG4 backbone. It clears excess Aβ with minimal effect on microglia so as to stimulate amyloid phagocytosis while limiting release of inflammatory cytokines as a way to avoid side effects such as vasogenic edema. In February 2015, Genentech started a Phase 1b study in 72 people with mild to moderate AD to compare three doses of intravenous crenezumab to placebo. Doses were not disclosed, but a 3-month double-blind course of monthly infusions of 60 mg/kg seems likely. In January 2019, Roche terminated both Phase 3 CREAD trials (see January 2019 news). Results for CREAD1, presented in March at the AD/PD conference in Lisbon, Portugal, were completely negative. The ARIA incidence with Crenezumab is low, but Crenezumab does not work at all on CDR-sum of boxes (CDR-SB), ADAS-Cog, and MMSE the drug and placebo curves are the same. The serum concentration of crenezumab increase in a dose-proportional manner between 15 and 120 mg/kg q4w. Total monomeric plasma Aβ(1-40) and Aβ(1-42) levels significantly increased after crenezumab administration, indicating target engagement.

A variety of tools are currently used to detect changes in cognition and other symptoms in patients with amyloid-related brain disorders. These tools may not only detect improvements or decline, but may also detect any synergistic effects resulting from the combination therapy proposed herein.

The Alzheimer's Disease (AD) Assessment Scale Cognitive Subscale (ADAS-Cog) is considered the gold standard for assessing the efficacy of anti-dementia treatments, and is familiar to those skilled in the art. Kueper et al., 2018, herein incorporated by reference with regard to such background teaching.

Clinical Global Impression of Change (CGIC) ratings are commonly used as tests of clinical meaningfulness in Alzheimer's disease (AD) trials. Overall global change or severity scales ratings, or activities of daily living scales, are required co-primary outcomes in regulatory or registration trials for drugs for dementia. The Alzheimer's Disease Cooperative Study-Clinician's Global Impression of Change rating (ADCS-CGIC) has been commonly used in 6 month AD clinical trials. It is the most frequently used example of a Clinician's Interview-Based Impression of Change with caregiver's input (i.e., a “CIBIC+”) and is familiar to those skilled in the art of clinical research in AD.

Digit Symbol Substitution Test (DSST) was developed as an experimental tool to understand human associative learning. Its clinical utility, owing to its brevity and high discriminant validity, was first recognized in the 1940s, and now the DSST is among the most commonly used tests in clinical neurology. It is a paper and pencil test and is familiar to those skilled in the art of clinical neurology.

### BRIEF SUMMARY

One embodiment described herein is, a method of treating an amyloid-related brain disorder comprising Alzheimer's disease or amyloid-related imaging abnormality (ARIA), the method comprising administrating a therapeutically effective amount of a compound of Formula I and an anti-amyloid beta antibody:

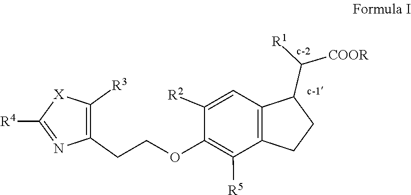

wherein
R is H, Na⁺, Li⁺, Ca⁺, K⁺, N⁺(C₁₋₆)₄, or C₁₋₆ alkyl;
R¹ is H, C₁₋₆ alkyl, C₃₋₆ cycloalkyl, or C₂₋₆ alkenyl, or C₁₋₆ alkoxy, each of which may be unsubstituted or substituted with fluoro, or phenyl which may be unsubstituted or substituted with R⁶, “c-2” is defined as the second carbon of the acetic acid portion of Formula I, and “c-1” is the first carbon of the indane group of Formula I;
R² is H, halo, or C₁₋₆ alkyl which may be unsubstituted or substituted with C₁₋₆ alkoxy, oxo, fluoro, or
R² is phenyl, furyl, thienyl, pyrrolyl, oxazolyl, thiazolyl, imidazolyl, pyrazolyl, pyridyl, pyrrolidinyl, piperidinyl, tetrahydropyranyl, piperazinyl, or morpholinyl, each of which may be unsubstituted or substituted with R⁶;
R³ is H, C₁₋₆ alkyl, or phenyl, which may be unsubstituted or substituted with R⁶;

### X is O or S;

R⁴ is phenyl, naphthyl, furyl, thienyl, pyrrolyl, oxazolyl, thiazolyl, pyrazolyl, pyridyl, pyrimidinyl, benzofuryl, benzothienyl, indolyl, indazolyl, benzoxazolyl, benzimidazolyl, quinolyl, isoquinolyl, or 1,4-benzodioxanyl, each of which may be unsubstituted or singlularly or multiply substituted with R⁶, or with phenyl, furyl, thienyl, pyrrolyl, each of which may be unsubstituted or singularly or multiply substituted with R⁶; or
R⁴ is C₁-C₆ alkyl or C₃-C₈ cycloalkyl, either of which may be unsubstituted or substituted with fluoro, oxo, or C₁-C₆ alkoxy which may be unsubstituted or substituted with C₁-C₆ alkoxy, or phenyl optionally substituted with R⁶, each of which may be substituted with phenyl, naphthyl, furyl, thienyl, pyrrolyl, each of which may be unsubstituted or further substituted with R⁶, or any C₁-C₆ alkyl may also be substituted with C₃-C₈ cycloalkyl or with phenoxy which may be unsubstituted or substituted with R⁶ or with phenyl, furyl, thienyl, pyrrolyl, each of which may be unsubstituted or substituted with R⁶, or
R⁵ is H, halo or C₁₋₆ alkyl optionally substituted with oxo;
R⁶ is halo, CF₃, C₁₋₆ alkyl optionally substituted with oxo or hydroxy, or C₁₋₆ alkoxy optionally substituted with fluoro;
wherein R³ may be attached to the heterocyclic moiety of the compound of Formula I at either the 4 or 5 position, and, accordingly, the remaining portion of the molecule will be attached at the remaining available carbon atom; or
a pharmaceutically acceptable salt, stereoisomer, enantiomer, racemate or combination thereof.

In one aspect, the anti-amyloid beta antibody comprises aducanumab, bapineuzumab, crenezumab, gantenerumab, or solanezumab.

In another aspect

### R¹ is H;

R² is H, halo, or C₁₋₆ alkyl which may be unsubstituted or substituted with C₁₋₆ alkoxy, oxo, fluoro;
R⁴ is phenyl, which may be unsubstituted or singularly or multiply substituted with R⁶; and
R⁵ is H, halo or C₁-C₆ alkyl optionally substituted with C₁-C₆ alkoxy, oxo, fluoro.

In another aspect

### R¹ is H;

R² is H or halo;
R³ is H or C₁₋₆ alkyl;
R⁴ is phenyl, which may be singularly or multiply substituted with R⁶;
R⁵ is H or halo;
R⁶ is halo, CF₃, C₁₋₆ alkyl or C₁₋₆ alkoxy; and
c-1′ has the S stereochemistry.

In another aspect

### R¹ is H;

R² is H or halo;
R³ is C₁₋₆ alkyl;

### X is O;

R⁴ is phenyl, which may be singularly or multiply substituted with R⁶;
R⁵ is H or halo;
R⁶ is halo, CF₃, C₁₋₆ alkyl or C₁₋₆ alkoxy; and
c-1′ has the S stereochemistry.

In another aspect,

### R¹ is H;

### R² is F;

R³ is C₁₋₆ alkyl;

### X is O;

R⁴ is phenyl, which may be singularly or multiply substituted with R⁶;

### R⁵ is F;

R⁶ is halo, CF₃, C₁₋₆ alkyl, C₁₋₆ alkoxy or C₁₋₆ alkyl; and
c-1′ has the S stereochemistry.

In another aspect

### R is H or Na;

### R¹ is H;

### R² is H;

R³ is C₁₋₆ alkyl;

### X is O;

R⁴ is phenyl, which may be singularly or multiply substituted with R⁶;

### R⁵ is H;

R⁶ is halo, CF₃, C₁₋₆ alkyl, C₁₋₆ alkoxy or C₁₋₆ alkyl; and
c-1′ has the S stereochemistry.

In another aspect

### R is H or Na;

### R¹ is H;

### R² is H;

R³ is C₁₋₆ alkyl;

### X is S;

R⁴ is phenyl, which may be singularly or multiply substituted with R⁶;

### R⁵ is H;

R⁶ is halo, CF₃, C₁₋₆ alkyl, C₁₋₆ alkoxy or C₁₋₆ alkyl; and
c-1′ has the S stereochemistry.

In another aspect, the compound is selected from the group consisting of

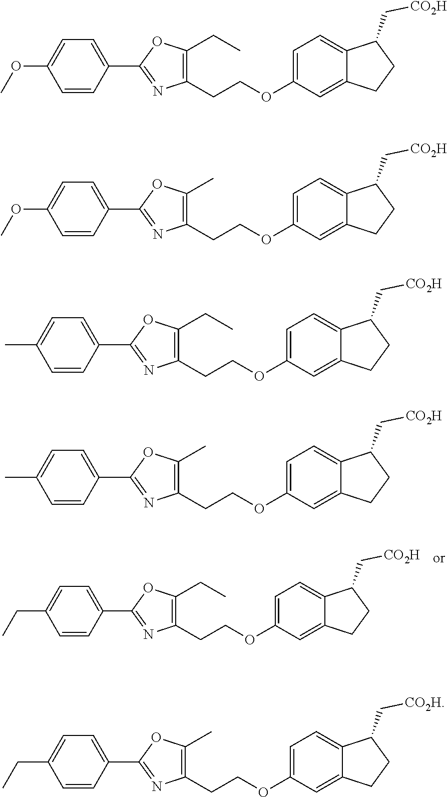 [SMILES: CCc1oc(-c2ccc(OC)cc2)nc1CCOc1ccc2c(c1)CC[C@H]2CC(=O)O ; COc1ccc(-c2nc(CCOc3ccc4c(c3)CC[C@H]4CC(=O)O)c(C)o2)cc1 ; CCc1oc(-c2ccc(C)cc2)nc1CCOc1ccc2c(c1)CC[C@H]2CC(=O)O ; Cc1ccc(-c2nc(CCOc3ccc4c(c3)CC[C@H]4CC(=O)O)c(C)o2)cc1 ; CCc1ccc(-c2nc(CCOc3ccc4c(c3)CC[C@H]4CC(=O)O)c(CC)o2)cc1 ; CCc1ccc(-c2nc(CCOc3ccc4c(c3)CC[C@H]4CC(=O)O)c(C)o2)cc1]

Another embodiment described herein is a method of reducing an amyloid-related brain disorder such as Alzheimer's disease or an amyloid-related imaging abnormality (ARIA), the method comprising administrating a therapeutically effective amount of:

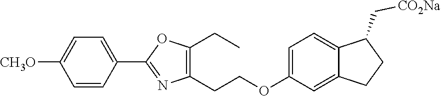

or
a pharmaceutically acceptable salt thereof and an anti-amyloid beta antibody, wherein the anti-amyloid beta antibody comprises aducanumab, bapineuzumab, crenezumab, gantenerumab, or solanezumab.

One aspect is a pharmaceutical composition comprising at least one pharmaceutically acceptable excipient and a therapeutically effective amount of any of the compounds described herein.

Another embodiment described herein is a method of treating a vasogenic edema comprising administrating a therapeutically effective amount of a compound of Formula I:

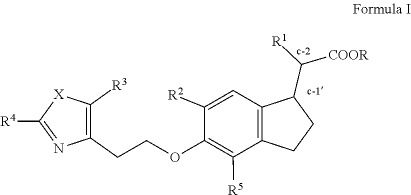

wherein
R is H, Na⁺, Li⁺, Ca⁺, K⁺, N⁺(C₁₋₆)₄, or C₁₋₆ alkyl;
R¹ is H, C₁₋₆ alkyl, C₃₋₆ cycloalkyl, or C₂₋₆ alkenyl, or C₁₋₆ alkoxy, each of which may be unsubstituted or substituted with fluoro, or phenyl which may be unsubstituted or substituted with R⁶, “c-2” is defined as the second carbon of the acetic acid portion of Formula I, and “c-1” is the first carbon of the indane group of Formula I;
R² is H, halo, or C₁₋₆ alkyl which may be unsubstituted or substituted with C₁₋₆ alkoxy, oxo, fluoro, or
R² is phenyl, furyl, thienyl, pyrrolyl, oxazolyl, thiazolyl, imidazolyl, pyrazolyl, pyridyl, pyrrolidinyl, piperidinyl, tetrahydropyranyl, piperazinyl, or morpholinyl, each of which may be unsubstituted or substituted with R⁶;
R³ is H, C₁₋₆ alkyl, or phenyl, which may be unsubstituted or substituted with R⁶;

### X is O or S;

R⁴ is phenyl, naphthyl, furyl, thienyl, pyrrolyl, oxazolyl, thiazolyl, pyrazolyl, pyridyl, pyrimidinyl, benzofuryl, benzothienyl, indolyl, indazolyl, benzoxazolyl, benzimidazolyl, quinolyl, isoquinolyl, or 1,4-benzodioxanyl, each of which may be unsubstituted or singlularly or multiply substituted with R⁶, or with phenyl, furyl, thienyl, pyrrolyl, each of which may be unsubstituted or singularly or multiply substituted with R⁶; or
R⁴ is C₁-C₆ alkyl or C₃-C₈ cycloalkyl, either of which may be unsubstituted or substituted with fluoro, oxo, or C₁-C₆ alkoxy which may be unsubstituted or substituted with C₁-C₆ alkoxy, or phenyl optionally substituted with R⁶, each of which may be substituted with phenyl, naphthyl, furyl, thienyl, pyrrolyl, each of which may be unsubstituted or further substituted with R⁶, or any C₁-C₆ alkyl may also be substituted with C₃-C₈ cycloalkyl or with phenoxy which may be unsubstituted or substituted with R⁶ or with phenyl, furyl, thienyl, pyrrolyl, each of which may be unsubstituted or substituted with R⁶, or
R⁵ is H, halo or C₁₋₆ alkyl optionally substituted with oxo;
R⁶ is halo, CF₃, C₁₋₆ alkyl optionally substituted with oxo or hydroxy, or C₁₋₆ alkoxy optionally substituted with fluoro;
wherein R³ may be attached to the heterocyclic moiety of the compound of Formula I at either the 4 or 5 position, and, accordingly, the remaining portion of the molecule will be attached at the remaining available carbon atom; or
a pharmaceutically acceptable salt, stereoisomer, enantiomer, racemate or combination thereof.

In one aspect, the compound is selected from the group consisting of

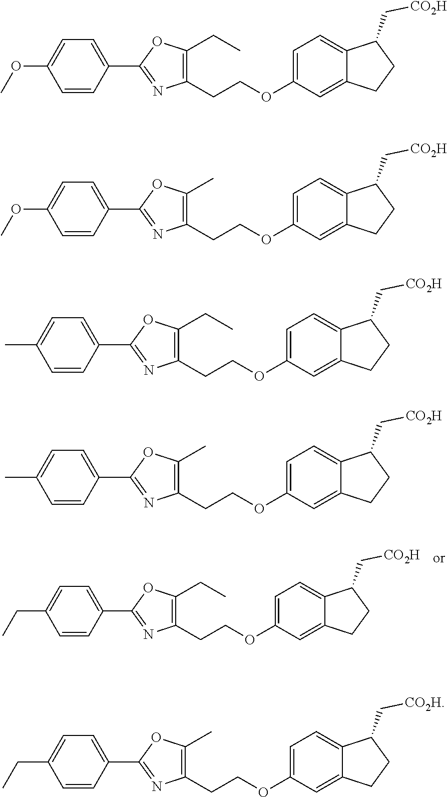 [SMILES: CCc1oc(-c2ccc(OC)cc2)nc1CCOc1ccc2c(c1)CC[C@H]2CC(=O)O ; COc1ccc(-c2nc(CCOc3ccc4c(c3)CC[C@H]4CC(=O)O)c(C)o2)cc1 ; CCc1oc(-c2ccc(C)cc2)nc1CCOc1ccc2c(c1)CC[C@H]2CC(=O)O ; Cc1ccc(-c2nc(CCOc3ccc4c(c3)CC[C@H]4CC(=O)O)c(C)o2)cc1 ; CCc1ccc(-c2nc(CCOc3ccc4c(c3)CC[C@H]4CC(=O)O)c(CC)o2)cc1 ; CCc1ccc(-c2nc(CCOc3ccc4c(c3)CC[C@H]4CC(=O)O)c(C)o2)cc1]

In another aspect, the method comprises administrating a therapeutically effective amount of:

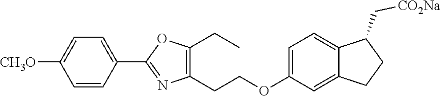

or
a pharmaceutically acceptable salt thereof.

Another embodiment described herein is, a method of treating cerebral amyloid angiopathy (CAA) comprising administrating a therapeutically effective amount of a compound of Formula I:

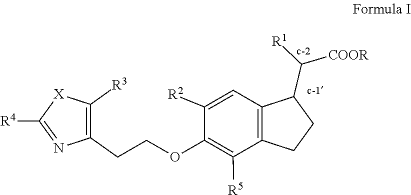

wherein
R is H, Na⁺, Li⁺, Ca⁺, K⁺, N⁺(C₁₋₆)₄, or C₁₋₆ alkyl;
R¹ is H, C₁₋₆ alkyl, C₃₋₆ cycloalkyl, or C₂₋₆ alkenyl, or C₁₋₆ alkoxy, each of which may be unsubstituted or substituted with fluoro, or phenyl which may be unsubstituted or substituted with R⁶, “c-2” is defined as the second carbon of the acetic acid portion of Formula I, and “c-1” is the first carbon of the indane group of Formula I;
R² is H, halo, or C₁₋₆ alkyl which may be unsubstituted or substituted with C₁₋₆ alkoxy, oxo, fluoro, or
R² is phenyl, furyl, thienyl, pyrrolyl, oxazolyl, thiazolyl, imidazolyl, pyrazolyl, pyridyl, pyrrolidinyl, piperidinyl, tetrahydropyranyl, piperazinyl, or morpholinyl, each of which may be unsubstituted or substituted with R⁶;
R³ is H, C₁₋₆ alkyl, or phenyl, which may be unsubstituted or substituted with R⁶;

### X is O or S;

R⁴ is phenyl, naphthyl, furyl, thienyl, pyrrolyl, oxazolyl, thiazolyl, pyrazolyl, pyridyl, pyrimidinyl, benzofuryl, benzothienyl, indolyl, indazolyl, benzoxazolyl, benzimidazolyl, quinolyl, isoquinolyl, or 1,4-benzodioxanyl, each of which may be unsubstituted or singlularly or multiply substituted with R⁶, or with phenyl, furyl, thienyl, pyrrolyl, each of which may be unsubstituted or singularly or multiply substituted with R⁶; or
R⁴ is C₁-C₆ alkyl or C₃-C₈ cycloalkyl, either of which may be unsubstituted or substituted with fluoro, oxo, or C₁-C₆ alkoxy which may be unsubstituted or substituted with C₁-C₆ alkoxy, or phenyl optionally substituted with R⁶, each of which may be substituted with phenyl, naphthyl, furyl, thienyl, pyrrolyl, each of which may be unsubstituted or further substituted with R⁶, or any C₁-C₆ alkyl may also be substituted with C₃-C₈ cycloalkyl or with phenoxy which may be unsubstituted or substituted with R⁶ or with phenyl, furyl, thienyl, pyrrolyl, each of which may be unsubstituted or substituted with R⁶, or
R⁵ is H, halo or C₁₋₆ alkyl optionally substituted with oxo;
R⁶ is halo, CF₃, C₁₋₆ alkyl optionally substituted with oxo or hydroxy, or C₁₋₆ alkoxy optionally substituted with fluoro;
wherein R³ may be attached to the heterocyclic moiety of the compound of Formula I at either the 4 or 5 position, and, accordingly, the remaining portion of the molecule will be attached at the remaining available carbon atom; or
a pharmaceutically acceptable salt, stereoisomer, enantiomer, racemate or combination thereof.

In another aspect, the compound is selected from the group consisting of

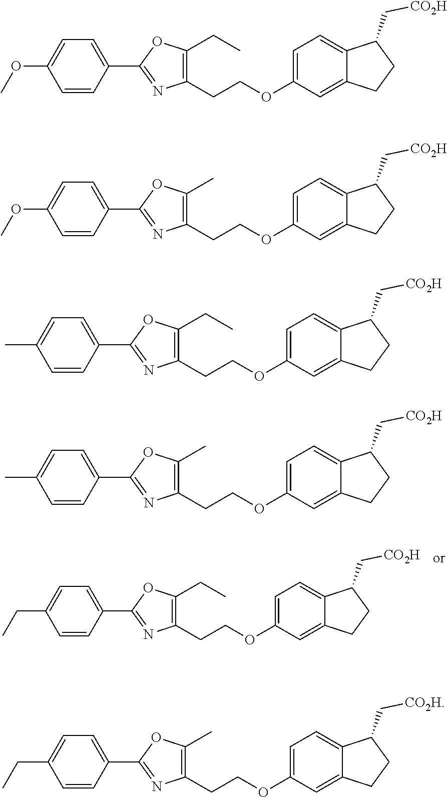 [SMILES: CCc1oc(-c2ccc(OC)cc2)nc1CCOc1ccc2c(c1)CC[C@H]2CC(=O)O ; COc1ccc(-c2nc(CCOc3ccc4c(c3)CC[C@H]4CC(=O)O)c(C)o2)cc1 ; CCc1oc(-c2ccc(C)cc2)nc1CCOc1ccc2c(c1)CC[C@H]2CC(=O)O ; Cc1ccc(-c2nc(CCOc3ccc4c(c3)CC[C@H]4CC(=O)O)c(C)o2)cc1 ; CCc1ccc(-c2nc(CCOc3ccc4c(c3)CC[C@H]4CC(=O)O)c(CC)o2)cc1 ; CCc1ccc(-c2nc(CCOc3ccc4c(c3)CC[C@H]4CC(=O)O)c(C)o2)cc1]

In another aspect, the method comprises administrating a therapeutically effective amount of:

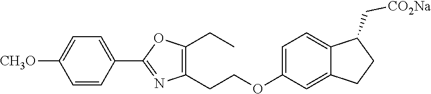

or
a pharmaceutically acceptable salt thereof.

Objects of the present disclosure will be appreciated by those of ordinary skill in

the art from a reading of the referenced patent literature and the Examples and the detailed description of the embodiments, which follow, such description being merely illustrative of the present disclosure.

### DETAILED DESCRIPTION

### A. Definitions

When describing the compounds, compositions, methods and processes of this disclosure, the following terms have the following meanings, unless otherwise indicated.

The terms “about” and “essentially” mean ±20 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit disclosures to only claiming the present disclosure with such comprising language. Any disclosure using the term comprising could be separated into one or more claims using “consisting” or “consisting of” claim language and is so intended.

References throughout this document to “one embodiment”, “certain embodiments”, and “an embodiment” or similar terms means that a particular feature, structure, or characteristic described in connection with the embodiment is included in at least one embodiment of the present disclosure. Thus, the appearances of such phrases in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs. In the event that there is a plurality of definitions for a term used herein, those in this section prevail unless stated otherwise.

“Alkyl” by itself or as part of another substituent refers to a hydrocarbon group which may be linear, cyclic, or branched or a combination thereof having the number of carbon atoms designated (i.e., C₁₋₈ means one to eight carbon atoms). Examples of alkyl groups include methyl, ethyl, n-propyl, isopropyl, n-butyl, t-butyl, isobutyl, sec-butyl, cyclohexyl, cyclopentyl, (cyclohexyl)methyl, cyclopropylmethyl, bicyclo[2.2.1]heptane, bicyclo[2.2.2]octane, etc.

“Alkoxy” refers to —O-alkyl. Examples of an alkoxy group include methoxy, ethoxy, n-propoxy etc.

“Alkenyl” refers to an unsaturated hydrocarbon group which may be linear, cyclic or branched or a combination thereof. Alkenyl groups with 2-8 carbon atoms are preferred. The alkenyl group may contain 1, 2 or 3 carbon-carbon double bonds. Examples of alkenyl groups include ethenyl, n-propenyl, isopropenyl, n-but-2-enyl, n-hex-3-enyl, cyclohexenyl, cyclopentenyl and the like. Alkenyl groups can be substituted or unsubstituted, unless otherwise indicated.

“Alkynyl” refers to an unsaturated hydrocarbon group which may be linear, cyclic or branched or a combination thereof. Alkynyl groups with 2-8 carbon atoms are preferred. The alkynyl group may contain 1, 2 or 3 carbon-carbon triple bonds. Examples of alkynyl groups include ethynyl, n-propynyl, n-but-2-ynyl, n-hex-3-ynyl and the like. Alkynyl groups can be substituted or unsubstituted, unless otherwise indicated.

“Aryl” refers to a polyunsaturated, aromatic hydrocarbon group having a single ring (monocyclic) or multiple rings (bicyclic), which can be fused together or linked covalently. Aryl groups with 6-10 carbon atoms are preferred, where this number of carbon atoms can be designated by C₆₋₁₀, for example. Examples of aryl groups include phenyl and naphthalene-1-yl, naphthalene-2-yl, biphenyl and the like. Aryl groups can be substituted or unsubstituted, unless otherwise indicated.

“Halo” or “halogen”, by itself or as part of a substituent refers to a chlorine, bromine, iodine, or fluorine atom.

“Heteroatom” is meant to include oxygen (O), nitrogen (N), sulfur (S) and silicon (Si).

“Haloalkyl”, as a substituted alkyl group, refers to a monohaloalkyl or polyhaloalkyl group, most typically substituted with from 1-3 halogen atoms. Examples include 1-chloroethyl, 3-bromopropyl, trifluoromethyl and the like.

“Heterocyclyl” refers to a saturated or unsaturated non-aromatic ring containing at least one heteroatom (typically 1 to 5 heteroatoms) selected from nitrogen, oxygen or sulfur. The heterocyclyl ring may be monocyclic or bicyclic. Preferably, these groups contain 0-5 nitrogen atoms, 0-2 sulfur atoms and 0-2 oxygen atoms. More preferably, these groups contain 0-3 nitrogen atoms, 0-1 sulfur atoms and 0-1 oxygen atoms. Examples of heterocycle groups include pyrrolidine, piperidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-dioxide, piperazine, pyran, pyridone, 3-pyrroline, thiopyran, pyrone, tetrahydrofuran, tetrahydrothiophene, quinuclidine and the like. Preferred heterocyclic groups are monocyclic, though they may be fused or linked covalently to an aryl or heteroaryl ring system.

“Heteroaryl” refers to an aromatic group containing at least one heteroatom, where the heteroaryl group may be monocyclic or bicyclic. Examples include pyridyl, pyridazinyl, pyrazinyl, pyrimidinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiazolyl, benzofuranyl, benzothienyl, indolyl, azaindolyl, azaindazolyl, quinolyl, isoquinolyl, isothiazolyl, pyrazolyl, indazolyl, pteridinyl, imidazolyl, triazolyl, tetrazolyl, oxazolyl, isoxazolyl, oxadiazolyl, thiadiazolyl, pyrrolyl, thiazolyl, furyl or thienyl. Preferred heteroaryl groups are those having at least one aryl ring nitrogen atom, such as quinolinyl, quinoxalinyl, purinyl, benzimidazolyl, benzopyrazolyl, benzotriazolyl, benzothiazolyl, indolyl, quinolyl, isoquinolyl and the like. Preferred 6-ring heteroaryl systems include pyridyl, pyridazinyl, pyrazinyl, pyrimidinyl, triazinyl and the like. Preferred 5-ring heteroaryl systems include isothiazolyl, pyrazolyl, imidazolyl, thienyl, furyl, triazolyl, tetrazolyl, oxazolyl, isoxazolyl, oxadiazolyl, thiadiazolyl, pyrrolyl, thiazolyl and the like.

Heterocyclyl and heteroaryl can be attached at any available ring carbon or heteroatom. Each heterocyclyl and heteroaryl may have one or more rings. When multiple rings are present, they can be fused together or linked covalently. Each heterocyclyl and heteroaryl must contain at least one heteroatom (typically 1 to 5 heteroatoms) selected from nitrogen, oxygen or sulfur. Preferably, these groups contain 0-5 nitrogen atoms, 0-2 sulfur atoms and 0-2 oxygen atoms. More preferably, these groups contain 0-3 nitrogen atoms, 0-1 sulfur atoms and 0-1 oxygen atoms. Heterocyclyl and heteroaryl groups can be substituted or unsubstituted, unless otherwise indicated. For substituted groups, the substitution may be on a carbon or heteroatom. For example, when the substitution is oxo (═O or —O⁻), the resulting group may have either a carbonyl (—C(O)—) or a N-oxide (—N⁺—O⁻).

Suitable substituents for substituted alkyl, substituted alkenyl, and substituted alkynyl include halogen, —CN, —CO₂R′, —C(O)R′, —C(O)NR′R″, oxo (═O or —O⁻), —OR′, —OC(O)R′, —OC(O)NR′R″—NO₂, —NR′C(O)R″, —NR′″C(O)NR′R″, —NR′R″, —NR′CO₂R″, —NR′S(O)R″, —NR′S(O)₂R′″, —NR′″S(O)NR′R″, —NR′″S(O)₂NR′R″, —SR′, —S(O)R′, —S(O)₂R′, —S(O)₂NR′R″, —NR′—C(NHR″)═NR′″, —SiR′R″R′″, —N₃, substituted or unsubstituted C₆₋₁₀ aryl, substituted or unsubstituted 5- to 10-membered heteroaryl, and substituted or unsubstituted 3- to 10-membered heterocyclyl. The number of possible substituents range from zero to (2m′+1), where m′ is the total number of carbon atoms in such radical.

Suitable substituents for substituted aryl, substituted heteroaryl and substituted heterocyclyl include halogen, —CN, —CO₂R′, —C(O)R′, —C(O)NR′R″, oxo (═O or —O⁻), —OR′, —OC(O)R′, —OC(O)NR′R″, —NO₂, —NR′C(O)R″, —NR′″C(O)NR′R″, —NR′R″, —NR′CO₂R″, —NR′S(O)R″, —NR′S(O)₂R″, —NR′″S(O)NR′R″, —NR′″S(O)₂NR′R″, —SR′, —S(O)R′, —S(O)₂R′, —S(O)₂NR′R″, —NR′—C(NHR″)═NR′″, —SiR′R″R′″, —N₃, substituted or unsubstituted C₁₋₈ alkyl, substituted or unsubstituted C₂₋₈ alkenyl, substituted or unsubstituted C₂₋₈ alkynyl, substituted or unsubstituted C₆₋₁₀ aryl, substituted or unsubstituted 5- to 10-membered heteroaryl, and substituted or unsubstituted 3- to 10-membered heterocyclyl. The number of possible substituents range from zero to the total number of open valences on the aromatic ring system.

As used above, R′, R″ and R′″ each independently refer to a variety of groups including hydrogen, substituted or unsubstituted C₁₋₈ alkyl, substituted or unsubstituted C₂₋₈ alkenyl, substituted or unsubstituted C₂₋₈ alkynyl, substituted or unsubstituted aryl, substituted or unsubstituted heteroaryl, substituted or unsubstituted heterocyclyl, substituted or unsubstituted arylalkyl, substituted or unsubstituted aryloxyalkyl. When R′ and R″ are attached to the same nitrogen atom, they can be combined with the nitrogen atom to form a 3-, 4-, 5-, 6-, or 7-membered ring (for example, —NR′R″ includes 1-pyrrolidinyl and 4-morpholinyl). Furthermore, R′ and R″, R″ and R′″, or R′ and R′″ may together with the atom(s) to which they are attached, form a substituted or unsubstituted 5-, 6-, or 7-membered ring.

The term “c-2” is defined as the second carbon of the acetic acid portion of Formula I, and is used to denote a chiral center when R1 is anything other than H; The term “c-1” is defined as the first carbon of the indane group of Formula I, which is a chiral center in all compounds of Formula I.

The term “optionally substituted” means that, unless indicated otherwise, the moiety so modified may have from one to up to the number of the substituents indicated, provided the resulting substitution is chemically feasible as recognized in the art. Each substituent may replace any H atom on the moiety so modified as long as the replacement is chemically possible and chemically stable. For example, a chemically unstable compound would be one where each of two substituents is bonded to a single C atom through each substituents heteroatom. Another example of a chemically unstable compound would be one where an alkoxy group is bonded to the unsaturated carbon of an alkene to form an enol ether. When there are two or more substituents on any moiety, each substituent is chosen independently of the other substituent so that, accordingly, the substituents can be the same or different.

When the 5- or 6-membered heterocyclic ring is attached to the rest of the molecule as a substituent, it becomes a radical. Examples of 5- or 6-membered heteroaryl ring radicals are furyl, pyrrolyl, thienyl, pyrazolyl, isoxazolyl, imidazolyl, oxazolyl, thiazolyl, isothiazolyl, triazolyl, thiadiazolyl, oxadiazolyl, pyridyl, pyrimidyl, pyridazinyl, pyrazinyl, triazinyl, and the like. Examples of partially unsaturated 5- or 6-membered heterocyclic ring radicals include dihydropyrano, pyrrolinyl, pyrazolinyl, imidazolinyl, dihydrofuryl, and the like. Examples of saturated 5- or 6-membered heterocyclic ring radicals include pyrrolidinyl, tetrahydropyridyl, piperidinyl, morpholinyl, tetrahydrofuryl, tetrahydrothienyl, piperazinyl, and the like. The point of attachment of the radical may be from any available C or N atom of the ring to the rest of the molecule. When the 5- or 6-membered heterocyclic ring is fused to another ring contained in the rest of the molecule, it forms a bicyclic ring. Examples of such 5- and 6-heterocyclic fused rings include pyrrolo, furo, pyrido, piperido, thieno, and the like. The point of fusion is at any available face of the heterocyclic ring and parent molecule.

The term “subject”, as used herein, means a mammalian subject (e.g., dog, cat, horse, cow, sheep, goat, monkey, etc.), and particularly human subjects (including both male and female subjects, and including neonatal, infant, juvenile, adolescent, adult and geriatric subjects, and further including various races and ethnicities including, but not limited to, white, black, Asian, American Indian and Hispanic).

“Pharmaceutically acceptable” carrier, diluent, or excipient is a carrier, diluent, or excipient compatible with the other ingredients of the formulation and not deleterious to the recipient thereof.

“Pharmaceutically-acceptable salt” refers to a salt which is acceptable for administration to a patient, such as a mammal (e.g., salts having acceptable mammalian safety for a given dosage regime). Such salts can be derived from pharmaceutically-acceptable inorganic or organic bases and from pharmaceutically-acceptable inorganic or organic acids, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Salts derived from pharmaceutically acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary, tertiary and quaternary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Salts derived from pharmaceutically-acceptable acids include acetic, ascorbic, benzenesulfonic, benzoic, camphosulfonic, citric, ethanesulfonic, fumaric, gluconic, glucoronic, glutamic, hippuric, hydrobromic, hydrochloric, isethionic, lactic, lactobionic, maleic, malic, mandelic, methanesulfonic, mucic, naphthalenesulfonic, nicotinic, nitric, pamoic, pantothenic, phosphoric, succinic, sulfuric, tartaric, p-toluenesulfonic and the like.

“Salt thereof” refers to a compound formed when the hydrogen of an acid is replaced by a cation, such as a metal cation or an organic cation and the like. Preferably, the salt is a pharmaceutically acceptable salt, although this is not required for salts of intermediate compounds which are not intended for administration to a patient.

In addition to salt forms, the present disclosure provides compounds which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

As used herein, “prevention”, “prevent”, and “preventing” refer to eliminating or reducing the incidence or onset of a disorder or disease as described herein, as compared to that which would occur in the absence of the measures taken.

As used herein, “therapeutically effective amount” refers to an amount amount sufficient to effect treatment when administered to a patient in need of treatment.

“Treating” or “treatment” as used herein refers to the treating or treatment of a disease or medical condition (such as a viral, bacterial or fungal infection or other infectious diseases, as well as autoimmune or inflammatory conditions) in a patient, such as a mammal (particularly a human or a companion animal) which includes ameliorating the disease or medical condition, i.e., eliminating or causing regression of the disease or medical condition in a patient; suppressing the disease or medical condition, i.e., slowing or arresting the development of the disease or medical condition in a patient; or alleviating or reducing the symptoms of the disease or medical condition in a patient.

Certain compounds of the present disclosure can exist in unsolvated forms as well as solvated forms, including hydrated forms. In general, both solvated forms and unsolvated forms are intended to be encompassed within the scope of the present disclosure. Certain compounds of the present disclosure may exist in multiple crystalline or amorphous forms (i.e., as polymorphs). In general, all physical forms are equivalent for the uses contemplated by the present disclosure and are intended to be within the scope of the present disclosure.

disclosuredisclosuredisclosure

The instant disclosure is directed to the use of novel compounds, which exert their biological activity as PPAR agonists, in combination with an amyloid-modifying therapies, particularly monoclonal antibodies directed against amyloid β for the reduction in frequency and severity of Amyloid Related Imaging Anomolies (ARIA) and amyoid related brain disorders. This novel concept requires that such a compound penetrates the blood brain barrier and has a pharmacological effect in the brain. That the compounds disclosed herein can achieve significant brain concentrations is surprising, as the general understanding in the literature is that carboxylic acids, which are negatively charged (anionic) at physiological pH values, are poorly brain penetrant. In fact, the ideal structure for blood brain penetration is generally considered to be positively charged (cationic at physiological pH) basic amine. As organic acids, it was also thought likely that indane acetic acids that did get into the brain would be rapidly eliminated from the brain by efflux transporters such as P glycoprotein (Pgp) or organic acid transporters (OATs) effectively limiting their brain to plasma concentrations. However, a study that measured rat brain concentrations of an exemplified compound, and showed that 12 hours after oral dosing, 35% of the amount in the plasma, was found in the brain. This is considered a substantial amount, and certainly enough to have a substantial pharmacological effect. In addition, an in vitro Pgp efflux experiment showed exemplified compounds were not substrates for the human Pgp transporter. Brain penetration was confirmed for an exemplified indane acetic acid in clinical trials by FDG-PET (F18 Fluorodeoxyglucose Positron Emission Tomography).

### B. Compounds

In one embodiment of the present disclosure described herein are the compounds of Formula I, which are PPAR delta and gamma dual agonists,

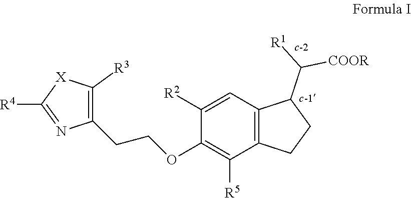

R is H, Na⁺, Li⁺, Ca⁺, K⁺, N⁺(C₁₋₆₎₄, or C₁₋₆ alkyl;

R¹ is H, C₁₋₆ alkyl, C₃₋₆ cycloalkyl, or C₂₋₆ alkenyl, or C₁₋₆ alkoxy, each of which may be unsubstituted or substituted with fluoro, or phenyl which may be unsubstituted or substituted with R⁶, “c-2” is defined as the second carbon of the acetic acid portion of Formula I, and “c-1” is the first carbon of the indane group of Formula I;

R² is H, halo, or C₁₋₆ alkyl which may be unsubstituted or substituted with C₁₋₆ alkoxy, oxo, fluoro,

or R² is phenyl, furyl, thienyl, pyrrolyl, oxazolyl, thiazolyl, imidazolyl, pyrazolyl, pyridyl, pyrrolidinyl, piperidinyl, tetrahydropyranyl, piperazinyl, or morpholinyl, each of which may be unsubstituted or substituted with R⁶;

R³ is H, C₁₋₆ alkyl, or phenyl, which may be unsubstituted or substituted with R⁶;

X is O or S;

R⁴ is phenyl, naphthyl, furyl, thienyl, pyrrolyl, oxazolyl, thiazolyl, pyrazolyl, pyridyl, pyrimidinyl, benzofuryl, benzothienyl, indolyl, indazolyl, benzoxazolyl, benzimidazolyl, quinolyl, isoquinolyl, or 1,4-benzodioxanyl, each of which may be unsubstituted or singlularly or multiply substituted with R⁶, or with phenyl, furyl, thienyl, pyrrolyl, each of which may be unsubstituted or singularly or multiply substituted with R⁶;

or R⁴ is C₁₋₆ alkyl or C₃₋₈ cycloalkyl, either of which may be unsubstituted or substituted with fluoro, oxo, or C₁-C₆ alkoxy which may be unsubstituted or substituted with C₁₋₆ alkoxy, or phenyl optionally substituted with R⁶, each of which may be substituted with phenyl, naphthyl, furyl, thienyl, pyrrolyl, each of which may be unsubstituted or further substituted with R⁶, or any C₁₋₆ alkyl may also be substituted with C₃₋₆₈ cycloalkyl or with phenoxy which may be unsubstituted or substituted with R⁶ or with phenyl, furyl, thienyl, pyrrolyl, each of which may be unsubstituted or substituted with R⁶, or

R⁵ is H, halo or C₁₋₆ alkyl optionally substituted with oxo; and

R⁶ is halo, CF₃, C₁₋₆ alkyl optionally substituted with oxo or hydroxy, or C₁₋₆ alkoxy optionally substituted with fluoro; and wherein R³ may be attached to the heterocyclic moiety of the compound of Formula I at either the 4 or 5 position, and, accordingly, the remaining portion of the molecule will be attached at the remaining available carbon atom; or a pharmaceutically acceptable salt thereof.

In one aspect, the compound of Formula I has structure as described above and R is potassium, sodium, calcium, magnesium, lysine, choline or meglumine salt thereof.

In another aspect R is H, R¹ is H, R² is H, R⁵ is H, R³ is C₁₋₆ alkyl, X is O, and R⁴ is a phenyl, singularly or multiply substituted with R⁶, wherein R⁶ is halo, CF₃, C₁-C₆ alkoxyl or C₁-C₆ alkyl, or a pharmaceutically acceptable salt thereof.

In another aspect R is H, R¹ is H, R² is H, R⁵ is H, R³ is C₁₋₆ alkyl, X is O, and R⁴ is a phenyl, singularly or multiply substituted with R⁶, wherein R⁶ is halo, CF₃, C₁-C₆ alkoxyl or C₁₋₆ alkyl, and the stereochemistry at C-1′ is defined as S, or a pharmaceutically acceptable salt thereof.

In another aspect R is H, R¹ is H, R² is H, R⁵ is H, R³ is C₁₋₆ alkyl, X is S, and R⁴ is a phenyl, singularly or multiply substituted with R⁶, wherein R⁶ is halo, CF₃, C₁₋₆ alkoxyl or C₁₋₆ alkyl, and the stereochemistry at C-1′ is defined as S, or a pharmaceutically acceptable salt thereof.

In yet another aspect R is H, R¹ is H, R² is F, R⁵ is H, R³ is C₁₋₆ alkyl, X is O, and R⁴ is a phenyl, singularly or multiply substituted with R⁶, wherein R⁶ is halo, CF₃, C₁₋₆ alkoxyl or C₁₋₆ alkyl, and the stereochemistry at C-1′ is defined as S, or a pharmaceutically acceptable salt thereof.

In another aspect R is H, R¹ is H, R² is H, R⁵ is F, or R² and R⁵ are F, R³ is C₁₋₆ alkyl, X is O, and R⁴ is a phenyl, singularly or multiply substituted with R⁶, wherein R⁶ is halo, CF₃, C₁₋₆ alkoxyl or C₁₋₆ alkyl, and the stereochemistry at C-1′ is defined as S, or a pharmaceutically acceptable salt thereof.

In another aspect R is H, R¹ is H, R² is H, R⁵ is H, R³ is C₁₋₆ alkyl, X is O, and R⁴ is a phenyl, singularly or multiply substituted with R⁶, wherein R⁶ is halo, CF₃, alkoxyl or C₁₋₆ alkyl, and the stereochemistry at C-1′ is defined as R, or a pharmaceutically acceptable salt thereof.

In another aspect the compound of Formula I is either the free acid or the potassium, sodium, calcium, magnesium, lysine, choline or meglumine salt of one of the following structures:

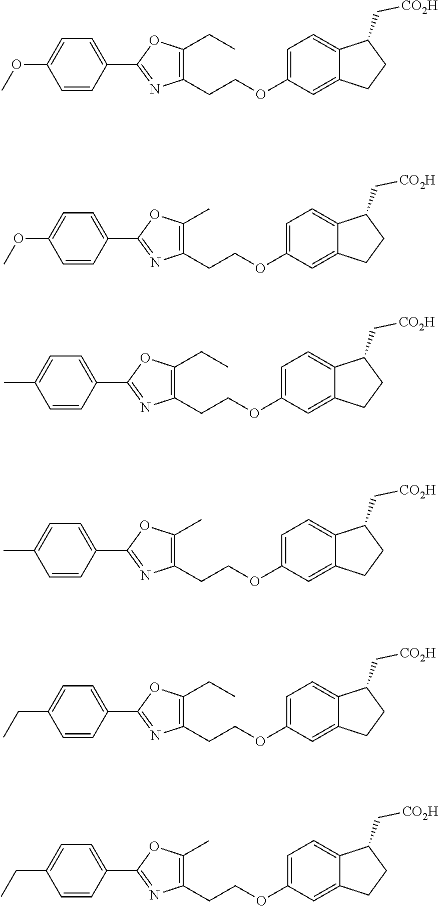 [SMILES: CCc1oc(-c2ccc(OC)cc2)nc1CCOc1ccc2c(c1)CC[C@H]2CC(=O)O ; COc1ccc(-c2nc(CCOc3ccc4c(c3)CC[C@H]4CC(=O)O)c(C)o2)cc1 ; CCc1oc(-c2ccc(C)cc2)nc1CCOc1ccc2c(c1)CC[C@H]2CC(=O)O ; Cc1ccc(-c2nc(CCOc3ccc4c(c3)CC[C@H]4CC(=O)O)c(C)o2)cc1 ; CCc1ccc(-c2nc(CCOc3ccc4c(c3)CC[C@H]4CC(=O)O)c(CC)o2)cc1 ; CCc1ccc(-c2nc(CCOc3ccc4c(c3)CC[C@H]4CC(=O)O)c(C)o2)cc1]

In another aspect the compound of Formula I is a potassium or sodium salt of the structures:

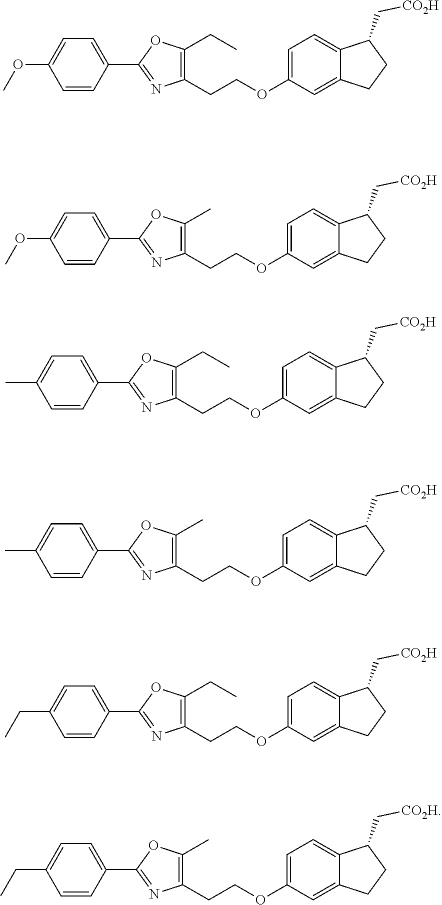 [SMILES: CCc1oc(-c2ccc(OC)cc2)nc1CCOc1ccc2c(c1)CC[C@H]2CC(=O)O ; COc1ccc(-c2nc(CCOc3ccc4c(c3)CC[C@H]4CC(=O)O)c(C)o2)cc1 ; CCc1oc(-c2ccc(C)cc2)nc1CCOc1ccc2c(c1)CC[C@H]2CC(=O)O ; Cc1ccc(-c2nc(CCOc3ccc4c(c3)CC[C@H]4CC(=O)O)c(C)o2)cc1 ; CCc1ccc(-c2nc(CCOc3ccc4c(c3)CC[C@H]4CC(=O)O)c(CC)o2)cc1 ; CCc1ccc(-c2nc(CCOc3ccc4c(c3)CC[C@H]4CC(=O)O)c(C)o2)cc1]

In another aspected described herein the compound is:

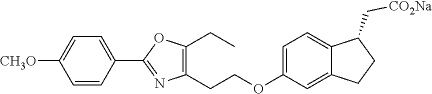

or,
a pharmaceutically acceptable salt thereof.

Exemplary compounds of Formula I are listed in Table 1 as the free acid, but may also be a pharmaceutically acceptable salt thereof.

TABLE 1
Illustrative Examples of Compounds of Formula I

| Entry | | | | | | |
|---|---|---|---|---|---|---|
| No. | R¹ | R² | R³ | R⁴ | R⁵ | X |
| 1 | H | H | Me | Ph | H | O |
| 2 | H | H | Me | 2-F-Ph | H | O |
| 3 | H | H | Me | 2-Cl Ph | H | O |
| 4 | H | H | Me | 2-Me Ph | H | O |
| 5 | H | H | Me | 3-F-Ph | H | O |
| 6 | H | H | Me | 3-Cl Ph | H | O |
| 7 | H | H | Me | 3-CF₃ Ph | H | O |
| 8 | H | H | Me | 3-Me Ph | H | O |
| 9 | H | H | Me | 3-MeO Ph | H | O |
| 10 | H | H | Me | 4-F-Ph | H | O |
| 11 | H | H | Me | 4-Cl Ph | H | O |
| 12 | H | H | Me | 4-CF₃ Ph | H | O |
| 13 | H | H | Me | 4-Me Ph | H | O |
| 14 | H | H | Me | 4-Et Ph | H | O |
| 15 | H | H | Me | 4-MeO Ph | H | O |
| 16 | H | H | Me | 4-EtO Ph | H | O |
| 17 | H | H | Me | 2,3-di-F Ph | H | O |
| 18 | H | H | Me | 2,4-di-F Ph | H | O |
| 19 | H | H | Me | 3,4-di-F Ph | H | O |
| 20 | H | H | Me | 2,6-di-F Ph | H | O |
| 21 | H | H | Me | 2,3-di-Cl Ph | H | O |
| 22 | H | H | Me | 3,4-di-Cl Ph | H | O |
| 23 | H | H | Me | 2,4-di-Cl Ph | H | O |
| 24 | H | H | Me | 2,6-di-Cl Ph | H | O |
| 25 | H | H | Me | 2,3-di-Me Ph | H | O |
| 26 | H | H | Me | 2,4-di-Me Ph | H | O |
| 27 | H | H | Me | 3,4-di-Me Ph | H | O |
| 28 | H | H | Me | 2,6-di-Me Ph | H | O |
| 29 | H | H | Me | 2,3-di-MeO Ph | H | O |
| 30 | H | H | Me | 2,4-di-MeO Ph | H | O |
| 31 | H | H | Me | 3,4-di-MeO Ph | H | O |
| 32 | H | H | Et | Ph | H | O |
| 33 | H | H | Et | 2-Cl Ph | H | O |
| 34 | H | H | Et | 2-Me Ph | H | O |
| 35 | H | H | Et | 3-F-Ph | H | O |
| 36 | H | H | Et | 3-Cl Ph | H | O |
| 37 | H | H | Et | 3-CF₃ Ph | H | O |
| 38 | H | H | Et | 3-Me Ph | H | O |
| 39 | H | H | Et | 3-MeO Ph | H | O |
| 40 | H | H | Et | 4-F-Ph | H | O |
| 41 | H | H | Et | 4-Cl Ph | H | O |
| 42 | H | H | Et | 4-CF₃ Ph | H | O |
| 43 | H | H | Et | 4-Me Ph | H | O |
| 44 | H | H | Et | 4-Et Ph | H | O |
| 45 | H | H | Et | 4-MeO Ph | H | O |
| 46 | H | H | Et | 4-EtO Ph | H | O |
| 47 | H | H | Et | 2,3-di-F Ph | H | O |
| 48 | H | H | Et | 2,4-di-F Ph | H | O |
| 49 | H | H | Et | 3,4-di-F Ph | H | O |
| 50 | H | H | Et | 2,6-di-F Ph | H | O |
| 51 | H | H | Et | 2,3-di-Cl Ph | H | O |
| 52 | H | H | Et | 2,4-di-Cl Ph | H | O |
| 53 | H | H | Et | 3,4-di-Cl Ph | H | O |
| 54 | H | H | Et | 2,6-di-Cl Ph | H | O |
| 55 | H | H | Et | 2,3-di-Me Ph | H | O |
| 56 | H | H | Et | 2,4-di-Me Ph | H | O |
| 57 | H | H | Et | 3,4-di-Me Ph | H | O |
| 58 | H | H | Et | 2,6-di-Me Ph | H | O |
| 59 | H | H | Et | 2,3-di-MeO Ph | H | O |
| 60 | H | H | Et | 2,4-di-MeO Ph | H | O |
| 61 | H | H | Et | 3,4-di-MeO Ph | H | O |
| 62 | H | H | iPr | Ph | H | O |
| 63 | H | H | iPr | 2-F Ph | H | O |
| 64 | H | H | iPr | 2-Cl Ph | H | O |
| 65 | H | H | iPr | 2-Me Ph | H | O |
| 66 | H | H | iPr | 2-MeO Ph | H | O |
| 67 | H | H | iPr | 3-F-Ph | H | O |
| 68 | H | H | iPr | 3-Cl Ph | H | O |
| 69 | H | H | iPr | 3-CF₃ Ph | H | O |
| 70 | H | H | iPr | 3-Me Ph | H | O |
| 71 | H | H | iPr | 3-MeO Ph | H | O |
| 72 | H | H | iPr | 4-F-Ph | H | O |
| 73 | H | H | iPr | 4-Cl Ph | H | O |
| 74 | H | H | iPr | 4-CF₃ Ph | H | O |
| 75 | H | H | iPr | 4-Me Ph | H | O |
| 76 | H | H | iPr | 4-Et Ph | H | O |
| 77 | H | H | iPr | 4-MeO Ph | H | O |
| 78 | H | H | iPr | 4-EtO Ph | H | O |
| 79 | H | H | iPr | 2,3-di-F Ph | H | O |
| 80 | H | H | iPr | 2,4-di-F Ph | H | O |
| 81 | H | H | iPr | 3,4-di-F Ph | H | O |
| 82 | H | H | iPr | 2,3-di-F Ph | H | O |
| 83 | H | H | iPr | 2,3-di-Cl Ph | H | O |
| 84 | H | H | iPr | 2,4-di-Cl Ph | H | O |
| 85 | H | H | iPr | 2,6-di-Cl Ph | H | O |
| 86 | H | H | iPr | 3,4-di-Cl Ph | H | O |
| 87 | H | H | iPr | 2,3-di-Me Ph | H | O |
| 88 | H | H | iPr | 2,4-di-Me Ph | H | O |
| 89 | H | H | iPr | 2,3-di-Me Ph | H | O |
| 90 | H | H | iPr | 2,3-di-Me Ph | H | O |
| 91 | H | H | iPr | 2,3-di-MeO Ph | H | O |
| 92 | H | H | iPr | 2,4-di-MeO Ph | H | O |
| 93 | H | H | iPr | 3,4-di-MeO Ph | H | O |
| 94 | Me | H | Et | 4-MeO Ph | H | O |
| 95 | Me | H | Et | 4-MeO Ph | H | S |
| 96 | H | H | Et | 4-MeO Ph | H | S |
| 97 | H | H | Me | 4-Et Ph | H | S |
| 98 | H | F | Et | 4-MeO Ph | H | O |
| 99 | H | H | Et | 4-MeO Ph | F | O |
| 100 | H | H | Et | 4-F-Ph | H | S |
| 101 | H | H | Et | 4-Cl Ph | H | S |
| 102 | H | H | Et | 4-CF₃ Ph | H | S |
| 103 | H | H | Et | 4-Me Ph | H | S |
| 104 | H | H | Et | 4-MeO Ph | H | S |
| 105 | H | H | Et | 4-EtO Ph | H | S |

Also contemplated herein are salt of any of the compounds described in the present disclosure. A salt of a compound disclosure may be prepared in situ during the final isolation and purification of a compound or by separately reacting the purified compound in its free base form with a suitable organic or inorganic acid and isolating the salt thus formed. Likewise, when the compounds described in the present disclosure contains a carboxylic acid moiety, (e.g., R═H), a salt of said compound may be prepared by separately reacting it with a suitable inorganic or organic base and isolating the salt thus formed. The term “pharmaceutically acceptable salt” refers to a relatively non-toxic, inorganic or organic acid addition salt of a compound of the present disclosure (see, e.g., Berge et al., J. Pharm. Sci. 66:1-19, 1977).

Representative salts of the compounds described herein include the conventional non-toxic salts and the quaternary ammonium salts, which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, undecanoate, and the like.

Base salts include, for example, alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups in the conjugate base may be quaternized with alkyl halides, e.g., C₁₋₉ alkyl halides such as methyl, ethyl, propyl, and butyl chlorides, bromides and iodides; dialkyl sulfates like dimethyl, diethyl, and dibutyl sulfate; and diamyl sulfates, C₁₀₋₄₀ alkyl halides such as decyl, lauryl, myristyl and strearyl chlorides, bromides and iodides; or aralkyl halides like benzyl and phenethyl bromides. In some embodiments, the salts are alkali salt such as sodium or potassium salt or an adduct with an acceptable nitrogen base such as meglumine (N-Methyl-d-glucamine) salt.

The esters of the compounds described disclosure herein are non-toxic, pharmaceutically acceptable esters, for example, alkyl esters such as methyl, ethyl, propyl, isopropyl, butyl, isobutyl, or pentyl esters. Additional esters such as, for example, methyl ester or phenyl-C₁-C₅ alkyl may be used. The compound described disclosure herein may be esterified by a variety of conventional procedures including reacting the appropriate anhydride, carboxylic acid, or acid chloride with the alcohol group of the compounds described in the present disclosure compound. The appropriate anhydride may be reacted with the alcohol in the presence of a base to facilitate acylation such as 1,8-bis[dimethylamino]naphthalene or N,N-dimethylaminopyridine. An appropriate carboxylic acid may be reacted with the alcohol in the presence of a dehydrating agent such as dicyclohexylcarbodiimide, 1-[3-dimethylaminopropyl]-3-ethylcarbodiimide, or other water soluble dehydrating agents which are used to drive the reaction by the removal of water, and optionally, an acylation catalyst. Esterification may also be effected using the appropriate carboxylic acid in the presence of trifluoroacetic anhydride and optionally, pyridine, or in the presence of N, N-carbonyldiimidazole with pyridine. Reaction of an acid chloride with the alcohol may be carried out with an acylation catalyst such as 4-DMAP or pyridine. One skilled in the art would readily know how to successfully carry out these, as well as other methods of esterification of alcohols.

Additionally, sensitive or reactive groups on the compound described in the present disclosure may need to be protected and deprotected during any of the above methods for forming esters. Protecting groups in general may be added and removed by conventional methods well known in the art (see, e.g., Chapter One of T. W. Greene and P. G. M. Wuts, Protective Groups in Organic Synthesis; Wiley: New York, (1999)).

The compounds described in the present disclosure may contain one or more asymmetric centers, depending upon the location and nature of the various substituents desired. Asymmetric carbon atoms may be present in the (R) or (S) configuration. Preferred isomers are those with the absolute configuration, which produces the compound of described in the present disclosure with the more desirable biological activity. In certain instances, asymmetry may also be present due to restricted rotation about a given bond, for example, the central bond adjoining two aromatic rings of the specified compounds.

Substituents on a ring may also be present in either cis or trans form, and a substituent on a double bond may be present in either Z or E form.

It is intended that all isomers (including enantiomers and diastereomers), either by nature of asymmetric centers or by restricted rotation as described above, as separated, pure or partially purified isomers or racemic mixtures thereof, be included within the scope of the instant disclosure. The purification of said isomers and the separation of said isomeric mixtures may be accomplished by standard techniques known in the art.

As described herein, compounds of the present disclosure may optionally be substituted with one or more substituents, such as are illustrated generally above, or as exemplified by particular classes, subclasses, and species of the disclosure. In general, the term “substituted” refers to the replacement of hydrogen radicals in a given structure with the radical of a specified substituent. Unless otherwise indicated, a substituted group may have a substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds.

Another embodiment of the disclosure includes a method of treating a an amyloid-related brain disorder comprising administering to a subject a therapeutically effective amount of any compound or a pharmaceutically acceptable salt thereof as described herein, or a composition as described herein.

### C. Pharmaceutical Compositions

According to another aspect of the present disclosure, pharmaceutical compositions of the compounds described herein are provided.

One aspect described herein, are pharmaceutical compositions comprising a therapeutically effective amount of any of the compounds described herein and at least one pharmaceutically acceptable excipient.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The pharmaceutical compositions for the administration of the compounds of this disclosure may conveniently be presented in unit dosage form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

Based on well-known assays used to determine the efficacy for treatment of conditions identified above in mammals, and by comparison of these results with the results of known medicaments that are used to treat these conditions, the effective dosage of the compounds of this disclosure can readily be determined for treatment of each desired indication. The amount of the active ingredient (e.g., compounds) to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered may generally range from about 0.0001 mg/kg to about 10 mg/kg, and preferably from about 0.001 mg/kg to about 10 mg/kg body weight per day. A unit dosage may contain from about 0.05 mg to about 500 mg of active ingredient, and may be administered one or more times per day. The daily dosage for administration by injection, including intravenous, intramuscular, subcutaneous, intranasal and parenteral injections, and use of infusion techniques may be from about 0.0001 mg/kg to about 10 mg/kg. The daily rectal dosage regimen may be from 0.0001 mg/kg to 10 mg/kg of total body weight. The transdermal concentration may be that required to maintain a daily dose of from 0.0001 mg/kg to 10 mg/kg. The daily intranasal dosage regimen may be from 0.0001 mg/kg to 10 mg/kg of total body weight.

Of course, the specific initial and continuing dosage regimen for each patient will vary according to the nature and severity of the condition as determined by the attending diagnostician, the activity of the specific compound employed, the age of the patient, the diet of the patient, time of administration, route of administration, rate of excretion of the drug, drug combinations, and the like. The desired mode of treatment and number of doses of a compound of the present disclosure may be ascertained by those skilled in the art using conventional treatment tests.

The compounds of the instant disclosure may be utilized to achieve the desired pharmacological effect by administration to a patient in need thereof in an appropriately formulated pharmaceutical composition. A patient, for the purpose of this disclosure, is a mammal, including a human, in need of treatment for a particular condition or disease. Therefore, the present disclosure includes pharmaceutical compositions which include a pharmaceutically acceptable carrier and a therapeutically effective amount of a compound. A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. A therapeutically effective amount of a compound is that amount which produces a result or exerts an influence on the particular condition being treated. The compounds described herein may be administered with a pharmaceutically-acceptable carrier using any effective conventional dosage unit forms, including, for example, immediate and timed release preparations, orally, parenterally, topically, intranasally or the like.

For oral administration, the compounds may be formulated into solid or liquid preparations such as, for example, capsules, pills, tablets, troches, lozenges, melts, powders, solutions, suspensions, or emulsions, and may be prepared according to methods known to the art for the manufacture of pharmaceutical compositions. The solid unit dosage forms may be a capsule which can be of the ordinary hard- or soft-shelled gelatin type containing, for example, surfactants, lubricants, and inert fillers such as lactose, sucrose, calcium phosphate, and corn starch.

In another embodiment, the compounds of the present disclosure may be tableted with conventional tablet bases such as lactose, sucrose, and cornstarch in combination with binders such as acacia, cornstarch, or gelatin; disintegrating agents intended to assist the break-up and dissolution of the tablet following administration such as potato starch, alginic acid, corn starch, and guar gum; lubricants intended to improve the flow of tablet granulation and to prevent the adhesion of tablet material to the surfaces of the tablet dies and punches, for example, talc, stearic acid, or magnesium, calcium or zinc stearate; dyes; coloring agents; and flavoring agents intended to enhance the aesthetic qualities of the tablets and make them more acceptable to the patient. Suitable excipients for use in oral liquid dosage forms include diluents such as water and alcohols, for example, ethanol, benzyl alcohol, and polyethylene alcohols, either with or without the addition of a pharmaceutically acceptable surfactant, suspending agent, or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent, and one or more preservatives. Suitable dispersing or wetting agents and suspending agents are exemplified by those already mentioned above. Additional excipients, for example, those sweetening, flavoring and coloring agents described above, may also be present.

The pharmaceutical compositions of this disclosure may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soybean and lecithin, (3) esters or partial esters derived from fatty acids and hexitol anhydrides, for example, sorbitan monooleate, and (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil, or coconut oil; or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol, or sucrose. Such formulations may also contain a demulcent, and preservative, flavoring and coloring agents.

The compounds of this disclosure may also be administered parenterally, that is, subcutaneously, intravenously, intramuscularly, or interperitoneally, as injectable dosages of the compound in a physiologically acceptable diluent with a pharmaceutical carrier which may be a sterile liquid or mixture of liquids such as water, saline, aqueous dextrose and related sugar solutions; an alcohol such as ethanol, isopropanol, or hexadecyl alcohol; glycols such as propylene glycol or polyethylene glycol; glycerol ketals such as 2,2-dimethyl-1,1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this disclosure are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures.

The parenteral compositions of this disclosure may typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The compounds of this disclosure may also be administered intranasally, as dosage of the compound in a physiologically acceptable diluent with a pharmaceutical carrier which may be a sterile liquid or mixture of liquids such as water, saline, aqueous dextrose and related sugar solutions; an alcohol such as ethanol, or hexadecyl alcohol; glycols such as propylene glycol or polyethylene glycol; glycerol ketals such as 2,2-dimethyl-1,1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, and isotonic sodium chloride solution. In addition, sterile fixed oils are conventionally employed as solvents or suspending media. For this purpose, any bland, fixed oil may be employed including synthetic mono or diglycerides. In addition, fatty acids such as oleic acid may be used in the preparation of injectables.

A composition of the disclosure may also be administered in the form of suppositories for rectal administration of the drug. These compositions may be prepared by mixing the drug (e.g., compound) with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present disclosure employs transdermal delivery devices (“patches”). Such transdermal patches may be used to provide continuous or discontinuous infusion of the compounds of the present disclosure in controlled amounts. The construction and use of transdermal patches for the delivery of pharmaceutical agents is well known in the art (see, e.g., U.S. Pat. No. 5,023,252, incorporated herein by reference). Such patches may be constructed for continuous, pulsatile, or on demand delivery of pharmaceutical agents.

It may be desirable or necessary to introduce the pharmaceutical composition to the patient via a mechanical delivery device. The construction and use of mechanical delivery devices for the delivery of pharmaceutical agents is well known in the art. For example, direct techniques for administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, incorporated herein by reference.

The compositions of the disclosure may also contain other conventional pharmaceutically acceptable compounding ingredients, generally referred to as carriers or diluents, as necessary or desired. Any of the compositions of this disclosure may be preserved by the addition of an antioxidant such as ascorbic acid or by other suitable preservatives. Conventional procedures for preparing such compositions in appropriate dosage forms can be utilized.

Commonly used pharmaceutical ingredients which may be used as appropriate to formulate the composition for its intended route of administration include: acidifying agents, for example, but are not limited to, acetic acid, citric acid, fumaric acid, hydrochloric acid, nitric acid; and alkalinizing agents such as, but are not limited to, ammonia solution, ammonium carbonate, diethanolamine, monoethanolamine, potassium hydroxide, sodium borate, sodium carbonate, sodium hydroxide, triethanolamine, or trolamine.

Other pharmaceutical ingredients include, for example, but are not limited to, adsorbents (e.g., powdered cellulose and activated charcoal); aerosol propellants (e.g., carbon dioxide, CCl₂F₂, F₂ClC—CClF₂ and CClF₃); air displacement agents (e.g., nitrogen and argon); antifungal preservatives (e.g., benzoic acid, butylparaben, ethylparaben, methylparaben, propylparaben, sodium benzoate); antimicrobial preservatives (e.g., benzalkonium chloride, benzethonium chloride, benzyl alcohol, cetylpyridinium chloride, chlorobutanol, phenol, phenylethyl alcohol, phenylmercuric nitrate and thimerosal); antioxidants (e.g., ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite); binding materials (e.g., block polymers, natural and synthetic rubber, polyacrylates, polyurethanes, silicones and styrene-butadiene copolymers); buffering agents (e.g., potassium metaphosphate, potassium phosphate monobasic, sodium acetate, sodium citrate anhydrous and sodium citrate dihydrate); carrying agents (e.g., acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection); chelating agents (e.g., edetate disodium and edetic acid); colorants (e.g., FD&C Red No. 3, FD&C Red No. 20, FD&C Yellow No. 6, FD&C Blue No. 2, D&C Green No. 5, D&C Orange No. 5, D&C Red No. 8, caramel and ferric oxide red); clarifying agents (e.g., bentonite); emulsifying agents (includes but are not limited to, acacia, cetomacrogol, cetyl alcohol, glyceryl monostearate, lecithin, sorbitan monooleate, polyethylene 50 stearate); encapsulating agents (e.g., gelatin and cellulose acetate phthalate); flavorants (e.g., anise oil, cinnamon oil, cocoa, menthol, orange oil, peppermint oil and vanillin); humectants (e.g., glycerin, propylene glycol and sorbitol); levigating agents (e.g., mineral oil and glycerin); oils (e.g., arachis oil, mineral oil, olive oil, peanut oil, sesame oil and vegetable oil); ointment bases (e.g., lanolin, hydrophilic ointment, polyethylene glycol ointment, petrolatum, hydrophilic petrolatum, white ointment, yellow ointment, and rose water ointment); penetration enhancers (transdermal delivery) (e.g., monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas); plasticizers (e.g., diethyl phthalate and glycerin); solvents (e.g., alcohol, corn oil, cottonseed oil, glycerin, isopropyl alcohol, mineral oil, oleic acid, peanut oil, purified water, water for injection, sterile water for injection and sterile water for irrigation); stiffening agents (e.g., cetyl alcohol, cetyl esters wax, microcrystalline wax, paraffin, stearyl alcohol, white wax and yellow wax); suppository bases (e.g., cocoa butter and polyethylene glycols (mixtures)); surfactants (e.g., benzalkonium chloride, nonoxynol 10, oxtoxynol 9, polysorbate 80, sodium lauryl sulfate and sorbitan monopalmitate); suspending agents (e.g., agar, bentonite, carbomers, carboxymethylcellulose sodium, hydroxyethyl cellulose, hydroxypropyl cellulose, hydroxypropyl methylcellulose, kaolin, methylcellulose, tragacanth and veegum); sweetening e.g., aspartame, dextrose, glycerin, mannitol, propylene glycol, saccharin sodium, sorbitol and sucrose); tablet anti-adherents (e.g., magnesium stearate and talc); tablet binders (e.g., acacia, alginic acid, carboxymethylcellulose sodium, compressible sugar, ethylcellulose, gelatin, liquid glucose, methylcellulose, povidone and pregelatinized starch); tablet and capsule diluents (e.g., dibasic calcium phosphate, kaolin, lactose, mannitol, microcrystalline cellulose, powdered cellulose, precipitated calcium carbonate, sodium carbonate, sodium phosphate, sorbitol and starch); tablet coating agents (e.g., liquid glucose, hydroxyethyl cellulose, hydroxypropyl cellulose, hydroxypropyl methylcellulose, methylcellulose, ethylcellulose, cellulose acetate phthalate and shellac); tablet direct compression excipients (e.g., dibasic calcium phosphate); tablet disintegrants (e.g., alginic acid, carboxymethylcellulose calcium, microcrystalline cellulose, polacrillin potassium, sodium alginate, sodium starch glycollate and starch); tablet glidants (e.g., colloidal silica, corn starch and talc); tablet lubricants (e.g., calcium stearate, magnesium stearate, mineral oil, stearic acid and zinc stearate); tablet/capsule opaquants (e.g., titanium dioxide); tablet polishing agents (e.g., carnuba wax and white wax); thickening agents (e.g., beeswax, cetyl alcohol and paraffin); tonicity agents (e.g., dextrose and sodium chloride); viscosity increasing agents (e.g., alginic acid, bentonite, carbomers, carboxymethylcellulose sodium, methylcellulose, povidone, sodium alginate and tragacanth); and wetting agents (e.g., heptadecaethylene oxycetanol, lecithins, polyethylene sorbitol monooleate, polyoxyethylene sorbitol monooleate, and polyoxyethylene stearate).

The compounds described herein may be administered as the sole pharmaceutical agent or in combination with one or more other pharmaceutical agents where the combination causes no unacceptable adverse effects. For example, compounds of this disclosure can be combined with known anti-oxidants, anti-obesity agents, insulin sensitizers, anti-fibrotics, anti-dyslipidemics, and the like, as well as with admixtures and combinations thereof.

The compounds described herein may also be utilized, in free acid or base form or in compositions, in research and diagnostics, or as analytical reference standards, and the like. Therefore, the present disclosure includes compositions which include an inert carrier and an effective amount of a compound identified by the methods described herein, or a salt or ester thereof. An inert carrier is any material which does not interact with the compound to be carried and which lends support, means of conveyance, bulk, traceable material, and the like to the compound to be carried. An effective amount of compound is that amount which produces a result or exerts an influence on the particular procedure being performed.

The compounds may be administered to subjects by any suitable route, including orally (inclusive of administration via the oral cavity), parenterally, by inhalation spray, topically, transdermally, rectally, nasally, sublingually, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, parenterally, transdermally or by inhalation spray.

It should also be understood that a specific dosage and treatment regimen for any particular patient will depend upon a variety of factors, including the activity of the specific compound employed, the age, body weight, general health, gender, diet, time of administration, rate of excretion, drug combination, and the judgment of the treating physician and the severity of the particular disease being treated. The amount of a compound of the present disclosure in the composition will also depend upon the particular compound in the composition.

The following examples are presented to illustrate the compositions described herein, but should not be construed as limiting the scope of the disclosure in any way.

Capsule Formulation
A capsule formula is prepared from:

| | |
|---|---|
| Compound of this disclosure | 10 mg |
| Starch | 109 mg |
| Magnesium stearate | 1 mg |

The components are blended, passed through an appropriate mesh sieve, and filled into hard gelatin capsules.

Tablet Formulation
A tablet is prepared from:

| | |
|---|---|
| Compound of this disclosure | 25 mg |
| Cellulose, microcrystalline | 200 mg |
| Colloidal silicon dioxide | 10 mg |
| Stearic acid | 5.0 mg |

The ingredients are mixed and compressed to form tablets. Appropriate aqueous and non-aqueous coatings may be applied to increase palatability, improve elegance and stability or delay absorption.

### Sterile IV Solution

A mg/mL solution of the desired compound of this disclosure is made using sterile, injectable water, and the pH is adjusted if necessary. The solution is diluted for administration with sterile 5% dextrose and is administered as an IV infusion.

Intramuscular suspension
The following intramuscular suspension is prepared:

| | |
|---|---|
| Compound of this disclosure | 50 mg/mL |
| Sodium carboxymethylcellulose | 5 mg/mL |
| TWEEN 80 | 4 mg/mL |
| Sodium chloride | 9 mg/mL |
| Benzyl alcohol | 9 mg/mL |

The suspension is administered intramuscularly.

### Hard Shell Capsules

A large number of unit capsules are prepared by filling standard two-piece hard galantine capsules each with powdered active ingredient, 150 mg of lactose, 50 mg of cellulose, and 6 mg of magnesium stearate.

sSoft Gelatin Capsules

A mixture of active ingredients in a digestible oil, such as soybean oil, cottonseed oil, or olive oil, is prepared and injected by means of a positive displacement pump into molten gelatin to form soft gelatin capsules containing the active ingredient. The capsules are washed and dried. The active ingredient can be dissolved in a mixture of polyethylene glycol, glycerin and sorbitol to prepare a water miscible medicine mix.

### Immediate Release Tablets/Capsules

These are solid oral dosage forms made by conventional and novel processes. These units are taken orally without water for immediate dissolution and delivery of the medication. The active ingredient is mixed in a liquid containing ingredient such as sugar, gelatin, pectin, and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

### Intranasal Formulation

These are liquid intranasal dosage forms made by conventional and novel processes. The active ingredient is mixed in a liquid containing ingredient such as sugar, gelatin, pectin.

### D. Methods of Use

Alzheimers Disease (AD) a chronic, progressive, neurodegenerative disease that is the cause of 60% to 70% of cases of dementia. The most common early symptom is short-term memory loss, and as the disease advances, symptoms can include language difficulties, disorientation, mood swings, loss of motivation, poor self care, and behavior issues. Over time, bodily functions are lost, ultimately leading to death. The average life expectancy following diagnosis is three to nine years. The cause of Alzheimer's disease is not well understood. About 70% of the risk is believed to be genetic. Type 2 Diabetes is also a significant risk factor, along with a history of head injuries, depression, or hypertension. The disease process is associated with amyloid plaques and tau neurofiburilary tangles in the brain. Thus, in one aspect of the present disclosure, Alzheimers Disease is an amyloid-related brain disorder. Diagnosis is based on a history of the illness and cognitive testing with medical imaging. Postmortem examination of brain tissue is needed for a definite diagnosis. Alzheimer's disease is characterized by loss of neurons and synapses in the cerebral cortex and certain subcortical regions. This loss results in gross atrophy of the affected regions, including degeneration in the temporal lobe and parietal lobe, and parts of the frontal cortex and cingulate gyrus. Studies using MRI and PET have documented reductions in the size of specific brain regions in people with AD as they progressed from mild cognitive impairment to Alzheimer's disease. Many different brain cell types such as astrocytes, microglia, oligodendrocytes and white matter and gray matter neurons are thought to play a role in AD neurodegeneration. There are approximately 30 million people worldwide with AD, mostly in people over 65 years of age, and leads to about 2 million deaths per year. There are no medications to stop or reverse the progression of AD.

Amyloid-related imaging abnormalities (ARIA) are abnormal differences seen in magnetic resonance imaging (MRI) of the brain of Alzheimer's disease patients, associated with amyloid-modifying therapies, particularly human monoclonal antibodies such as aducanumab. Thus, in one aspect of the present disclosure, an amyloid-related brain disorder comprises the presence of an amyloid-related imaging abnormality (ARIA). There are two types of ARIA: vasogenic edema and sulcal effusions (ARIA-E) and hemosiderin deposits (ARIA-H).

ARIA-E or extracellular brain edema, also called vasogenic edema, is an aberrant increase in the permeability of the blood-brain barrier. The breakdown of the normally tight endothelial junctions that make up the blood-brain barrier allows extravasation of fluid, ions, and plasma proteins into the brain which is detected by MRI. In one aspect of the present disclosure, vasogenic edema is an amyloid-related vasogenic edema. In another aspect of the disclosure, general vasogenic edema that is not the result of an amyloid-related brain disorder is contemplated as a non-amyloid vasogenic edema.

ARIA-H represents a spectrum of MRI findings due to haemosiderin deposition again resulting from breakdown of vascular integrity. Haemosiderin is an iron containing ferritin complex that forms when blood leaves a ruptured blood vessel and the iron containing hemoglobin is released into the extracellular space. Macrophages engulf the hemoglobin to degrade it, producing iron containing hemosiderin, which can be detected by MRI. It is thought that cerebral micro-hemorrhages, cerebral edemas, or vasogenic edemas, are related to amyloid burden and to the removal of amyloid plaques from cerebral blood vessels by amyloid targeted antibodies. Amyloid-related imaging abnormalities (ARIA) have been reported in AD patients treated with bapineuzumab, a humanized monoclonal antibody to amyloid-β. ARIA includes MRI signal abnormalities suggestive of ARIA-E and ARIA-H. Over the past decade since amyloid-modifying therapeutic agents have entered AD clinical trials, the occurrence of MRI abnormalities has required careful consideration by academic investigators, pharmaceutical companies and regulatory authorities.

Cerebral amyloid angiopathy (CAA) is the accumulation of amyloidogenic proteins, most often amyloid β (Aβ), in cerebral blood vessel walls, leading to a weakened vasculature and thereby creating a major risk for intracerebral hemorrhages (ICH). Thus, in one aspect of the present disclosure, CAA is an amyloid-related brain disorder. Several types of hereditary disorders exist that result in CAA, caused by missense mutations within the Aβ precursor protein gene. However, CAA most frequently occurs sporadically and is observed in cognitively normal elderly, but also frequently in patients with AD. In patients with AD, CAA is tightly linked to the development of ARIA, a frequently occurring side-effect of anti-Aβ immunotherapy defined by neuroimaging (eg, ≈40% of AD patients treated with aducanumab develop ARIA). Jäkel et al., 2021, herein incorporated by reference with regard to such background teaching. Patients with CAA may present with a broad clinical spectrum, including cognitive decline, lobar ICH, and transient focal neurological episodes (recurrent, stereotyped, transient episodes of smoothly spreading paraesthesias, numbness or weakness, lasting typically seconds to minutes, usually resolving over a similar period). A rare complication of the disease is CAA-related inflammation, characterized by headache, seizures, behavioral change, focal neurological signs, impaired consciousness. CAA is a major cause of primary intracerebral hemorrhage. Cerebrovascular deposition of the ß-amyloid peptide also appears to cause ischemic brain injury. Advanced CAA is associated with various neuropathological or neuroimaging markers of non-hemorrhagic tissue injury, including microinfarcts, white matter lesions, and altered diffusion-tensor properties. These multifocal lesions likely contribute to the association between advanced CAA and cognitive impairment observed in elderly individuals. A definite diagnosis of CAA can only be obtained by post mortem neuropathological assessment of brain tissue. For the diagnosis of CAA during life, criteria have been developed, which make use of MRI to enable the diagnosis of “probable” or “possible” CAA. These “Boston criteria” are based on two CAA-related imaging markers: strictly lobar cerebral microbleeds (small brain bleeds restricted to cortical and subcortical regions of the brain), and cortical superficial siderosis (deposition of blood breakdown products in the cortical sulci over the convexity of the cerebral hemispheres). Systematic review and meta-analysis of the literature, provides reliable estimates of the prevalence of CAA pathology and MRI imaging markers of CAA in AD patients, the general elderly population, cognitively normal elderly, and in patients with lobar Intra Cerebral Hemorrhage) ICH. Almost a quarter of the elderly general population has moderate-to-severe CAA pathology. Also, in AD patients (48%) and patients with lobar ICH (57%), CAA is highly prevalent. Since CAA is associated with the development of ARIA in anti-Aβ immunotherapy and with a growing spectrum of clinical symptoms, there is a growing awareness of the high prevalence of CAA.

The in vivo diagnosis of cerebral amyloid angiopathy (CAA) is currently based on MRI, which largely rely on hemorrhagic features on brain magnetic resonance imaging. FDG (¹⁸F-fluoro-deoxy-D-glucose) PET (positron emission tomography), has been studied to improve in vivo diagnosis of CAA. Studies have shown that FDG uptake is reduced in posterior cortical areas, particularly the primary occipital cortex, including the including the visual cortex, which pathologically bear the brunt of vascular Aβ deposition. Significant hypometabolism (P range, 0.047 to <0.0001) has been observed in the posterior cortical areas, including the superior and inferior parietal, primary visual, lateral occipital, lateral temporal, precuneus, and posterior cingulate regions of interest, in CAA patients. This suggests that FDG PET data is relevant to the study of CAA and ARIAs.

One aspect of the present disclosure are antibodies directed to amyloid beta and are anti-amyloid beta antibodies. The present disclosure contemplates the use of any anti-amyloid beta antibody, including the specific ones described herein.

ADUHELM™ (aducanumab) is an amyloid beta-directed antibody indicated for the treatment of Alzheimer's disease sold by Biogen. Treatment with ADUHELM™ is for patients with mild cognitive impairment or mild dementia stage of disease, the population in which treatment was initiated in clinical trials. This indication is approved under accelerated approval based on reduction in amyloid beta plaques observed in patients treated with aducanumab. Titration of the aducanumab dose is required for treatment initiation. The recommended maintenance dosage is 10 mg/kg administered as an intravenous infusion over approximately one hour every four weeks. Patients obtain a recent (within one year) brain MRI prior to initiating treatment. MRIs should be obtained prior to the 7th and 12th infusions. If radiographic severe ARIA-H is observed, treatment may be continued with caution only after a clinical evaluation and a follow-up MRI demonstrates radiographic stabilization (i.e., no increase in size or number of ARIA-H). Dilution in 100 ml of 0.9% Sodium Chloride Injection, USP, is required prior to administration. Doses can be 170 mg/1.7 mL (100 mg/mL) solution in a single-dose vial or 300 mg/3 mL (100 mg/mL) solution in a single-dose vial. ADUHELM™ can cause amyloid related imaging abnormalities-edema (ARIA-E), which can be observed on MRI as brain edema or sulcal effusions, and amyloid related imaging abnormalities hemosiderin deposition (ARIA-H), which includes microhemorrhage and superficial siderosis. ARIA-E was observed in 35% of patients treated with ADUHELM™ 10 mg/kg, compared to 3% of patients on placebo. The incidence of ARIA-E was higher in apolipoprotein E ε4 (ApoE ε4) carriers than in ApoE ε4 non-carriers (42% and 20%, respectively). The majority of ARIA-E radiographic events occurred early in treatment (within the first 8 doses), although ARIA can occur at any time. Among patients treated with a planned dose of ADUHELM™ 10 mg/kg who had ARIA-E, the maximum radiographic severity was mild in 30%, moderate in 58%, and severe in 13% of patients. Resolution occurred in 68% of ARIA-E patients by 12 weeks, 91% by 20 weeks, and 98% overall after detection. 10% of all patients who received ADUHELM™ 10 mg/kg had more than one episode of ARIA-E. ARIA-H associated with the use of ADUHELM™ 10 mg/kg was observed in 21% of patients treated with ADUHELM™ 10 mg/kg, compared to 1% of patients on placebo. Clinical symptoms were present in 24% of patients treated with ADUHELM™ 10 mg/kg who had an observation of ARIA (-E and/or -H), compared to 5% of patients on placebo. The most common symptom in patients treated with ADUHELM™ 10 mg/kg with ARIA was headache (13%). Other frequent symptoms were confusion/delirium/altered mental status/disorientation (5%), dizziness/vertigo (4%), visual disturbance (2%), and nausea (2%). Serious symptoms associated with ARIA were reported in 0.3% of patients treated with ADUHELM™ 10 mg/kg. Clinical symptoms resolved in the majority of patients (88%) during the period of observation. Enhanced clinical vigilance for ARIA is recommended during the first 8 doses of treatment with ADUHELM™, particularly during titration, as this is the time the majority of ARIA was observed.

Bapineuzumab is a humanized monoclonal antibody may have potential therapeutic value for the treatment of AD. Bapineuzumab lowered key biomarkers of AD: amyloid brain plaque and hyperphosphorylated tau protein in CSF, but failed to produce significant cognitive improvements in patients in two major trials, despite lowering. Bapineuzumab is an antibody to the beta-amyloid (Aβ) plaques that are believed to underlie AD neuropathology. It binds to the extreme N-terminal 5 residues of Aβ peptide. In a previous clinical trial targeting human beta amyloid, (AN-1792), demonstrated positive outcomes with removal of plaques, but 6% but was stopped for safety reasons. Bapineuzumab was the first beta-amyloid antibody to be found to cause amyloid-related imaging abnormalities (ARIA), including an accumulation of fluid in brain tissue (ARIA-E) in patients receiving the highest dose. No health risks were found in subjects receiving either 0.5 or 1 mg of bapineuzumab. Common doses of Bapineuzumab include: doses of bapineuzumab (0.15 mg/kg, 0.5 mg/kg, 1.0 mg/kg and 2.0 mg/kg). Dosing with Bapineuzumab can be either subcutaneous or intravenous.

Gantenerumab is a fully human IgG1 antibody which binds with sub-nanomolar affinity to a conformational epitope on Aβ fibrils. It binds to both N-terminal and central amino acids of Aβ. Gantenerumab is thought to disassemble and degrade amyloid plaques in the CNS by recruiting microglia and activating phagocytosis. Gantenerumab preferentially interacts with aggregated brain Aβ, both parenchymal and vascular. The antibody elicits phagocytosis of human Aβ deposits in AD brain slices co-cultured with human macrophages. In APP/PS-1 transgenic mice, gantenerumab binds to cerebral Aβ, reduces small plaques by recruiting microglia, and prevents new plaque formation. Gantenerumab does not alter plasma Aβ. Gantenerumab is generally safe and well-tolerated, but amyloid-related imaging abnormalities (ARIA) are observed in perhaps one third of patients at the highest dose, including both focal areas of inflammation or vasogenic edema on MRI scans in brain areas with the most amyloid reduction. Roche started a Phase 2 trial of 105 or 225 mg gantenerumab injected subcutaneously once a month into 360 participants, and in 2012 expanded the study to a Phase 2/3 registration trial of 799 people. Called Scarlet Road, this multinational, 159-center study of gantenerumab's effect on cognition and function in prodromal Alzheimer's disease delivered treatment for two years with the option of a two-year extension. The trial was stopped in December of 2014 based on an interim futility analysis. A Phase 3 trial with Gantenerumab dosed as high as 1200 mg subcutaneously. Gantenerumab lowered brain amyloid by an average of 59 centiloid on florbetapir PET, with half of the 28 participants who reached this timepoint falling below the threshold for amyloid positivity, and the rest on trajectory to do so. About one-third of participants in the extension studies developed ARIA-E. Clinical trial data from Roche shows that Gantenerumab treatment led to a large reduction in amyloid plaque as per PiB PET scans, and it normalized CSF Aβ42. Gantenerumab normalized elevated levels of CSF total tau and p-tau181, and slowed the rise of CSF neurofilament light. No clinical trial with Gantenerumab has shown a statistically significant improvement in cognition. In further analyses of the two-year open-label extension data Roche showed that ARIA tends to occurs in hotspots of rapid amyloid removal, but is not required for amyloid clearance across the brain. In current studies use monthly shots of 120 mg, ramping up to a target dose of 255 mg weekly.

Solanezumab is a humanized monoclonal IgG1 antibody directed against the mid-domain of the Aβ peptide. It recognizes soluble monomeric, not fibrillar, Aβ. The therapeutic rationale is that it may exert benefit by sequestering Aβ, shifting equilibria between different species of Aβ, and removing small soluble species of Aβ that are directly toxic to synaptic function. In Phase 2, trials administering 100 to 1,600 mg per month of solanezumab for 12 weeks, and monitoring for safety and biomarker effects for one year, confirmed the antibody's safety and tolerability. Phase 2 showed dose-dependent increases of various Aβ species in plasma and CSF but no effects on the ADAS-Cog, or indication of clinical benefit. In July 2013, Lilly started EXPEDITION-3, a 39-center Phase 3 trial in 2,100 patients with mild AD and demonstrated brain amyloid burden. In March 2016, Lilly announced that it would change the primary outcome for this trial. The trial used ADAS-Cog as a single primary and ADCS-iADL as a secondary outcome. On Nov. 23, 2016, Lilly announced, based on top-line results, that solanezumab had missed its primary endpoint in this trial. Primary and secondary outcome results were trending in the direction of a treatment benefit, but effects were small and fell short of statistical significance. Clinical biomarker data showed solanezumab treatment caused a steep increase in CSF Aβ42, indicating target engagement. Solanezumab did not alter tau biomarkers, and changed CSF neurofilament light concentrations for the worse. Changes in plasma amyloid-beta were similar to those seen in previous studies, and the differences between treatment and placebo groups were statistically significant. Changes in amyloid deposition as measured by positron emission tomography (PET) imaging did not reach statistical significance between treatment and placebo groups. The incidence of vasogenic edema (ARIA-E or amyloid-related imaging abnormality-edema/effusions) was approximately 0.1 percent of patients treated with solanezumab and 0.3 percent of patients on placebo.

Crenezumab is a passive immunotherapy approach in which patients are treated with monoclonal antibodies that specifically recognize Aβ peptides. Crenezumab recognizes multiple forms of aggregated Aβ, including oligomeric and fibrillar species and amyloid plaques with high affinity, and monomeric Aβ with low affinity. It uses a humanized antibody with an IgG4 backbone. It clears excess Aβ with minimal effect on microglia so as to stimulate amyloid phagocytosis while limiting release of inflammatory cytokines as a way to avoid side effects such as vasogenic edema. In February 2015, Genentech started a Phase 1b study in 72 people with mild to moderate AD to compare three doses of intravenous crenezumab to placebo. Doses were not disclosed, but a 3-month double-blind course of monthly infusions of 60 mg/kg seems likely. In January 2019, Roche terminated both Phase 3 CREAD trials (see January 2019 news). Results for CREAD1, presented in March at the AD/PD conference in Lisbon, Portugal, were completely negative. The ARIA incidence with Crenezumab is low, but Crenezumab does not work at all on CDR-sum of boxes (CDR-SB), ADAS-Cog, and MMSE the drug and placebo curves are the same. The serum concentration of crenezumab increase in a dose-proportional manner between 15 and 120 mg/kg q4w. Total monomeric plasma Aβ(1-40) and Aβ(1-42) levels significantly increased after crenezumab administration, indicating target engagement.

A variety of tools are currently used to detect changes in cognition and other symptoms in patients with amyloid-related brain disorders. These tools may not only detect improvements or decline, but may also detect any synergistic effects resulting from the combination therapy proposed herein.

The Alzheimer's Disease (AD) Assessment Scale Cognitive Subscale (ADAS-Cog) is considered the gold standard for assessing the efficacy of anti-dementia treatments, and is familiar to those skilled in the art. Kueper et al., 2018, herein incorporated by reference with regard to such background teaching.

Clinical Global Impression of Change (CGIC) ratings are commonly used as tests of clinical meaningfulness in Alzheimer's disease (AD) trials. Overall global change or severity scales ratings, or activities of daily living scales, are required co-primary outcomes in regulatory or registration trials for drugs for dementia. The Alzheimer's Disease Cooperative Study-Clinician's Global Impression of Change rating (ADCS-CGIC) has been commonly used in 6 month AD clinical trials. It is the most frequently used example of a Clinician's Interview-Based Impression of Change with caregiver's input (i.e., a “CIBIC+”) and is familiar to those skilled in the art of clinical research in AD.

Digit Symbol Substitution Test (DSST) was developed as an experimental tool to understand human associative learning. Its clinical utility, owing to its brevity and high discriminant validity, was first recognized in the 1940s, and now the DSST is among the most commonly used tests in clinical neurology. It is a paper and pencil test and is familiar to those skilled in the art of clinical neurology.

In still other embodiments, the present disclosure is directed to a method of treating an amyloid-related brain disorder comprising a compound or composition of the disclosure either alone or in combination with a second therapeutic agent, wherein said second therapeutic agent may be an anti-amyloid beta antibody. Without being bound by any theory, it is believed that when the compounds of the present disclosure are combined with an anti-amyloid beta antibody as described herein, the combination of both results in a synergistic effect that is more than just the additive result of each therapeutic individually. Furthermore, it may be possible to modify the dose of one therapeutic by reducing the doses of the other, thereby reducing the potential side effects of each therapeutic individually.

When used in combination, the practitioner can administer a combination of the compound or composition of the present disclosure and a second therapeutic agent. Also, the compound or composition and the second therapeutic agent can be administered sequentially, in any order.

The compounds and compositions of the present disclosure can be combined with other compounds and compositions having related utilities to prevent and treat the condition or disease of interest. Selection of the appropriate agents for use in combination therapies can be made one of ordinary skill in the art. The combination of therapeutic agents may act synergistically to effect the treatment or prevention of the various disorders. Using this approach, one may be able to achieve therapeutic efficacy with lower dosages of each agent, thus reducing the potential for adverse side effects.

In another aspect, the compounds and compositions of the present disclosure may be administered with an antibody. In one aspect the antibody may be an anti-amyloid antibody. In another aspect, the antibody comprises aducanumab, bapineuzumab, crenezumab, gantenerumab, or solanezumab.

Depending on the individual medicaments utilized in a combination therapy for simultaneous administration, they may be formulated in combination (where a stable formulation may be prepared and where desired dosage regimes are compatible) or the medicaments may be formulated separately (for concomitant or separate administration through the same or alternative routes). For separate administration the compound and antibody may be administered in any order.

In some embodiments, the subject of the present disclosure may be examined by MRI to investigate the incidence and severity of the ARIAs commonly observed in treatment of Alzheimer's patients with amyloid-modifying therapies, particularly monoclonal antibodies directed against amyloid β selected from the following list: aducanumab, bapineuzumab, Crenezumab, gantenerumab, or solanezumab.

### E. Example

Embodiments of the present disclosure will now be described by way of example only with respect to the following non-limiting examples.

The particular process to be utilized in the preparation of the compounds of this disclosure depends upon the specific compound desired. Such factors as the selection of the specific X moiety, and the specific substituents possible at various locations on the molecule, all play a role in the path to be followed in the preparation of the specific compounds of this disclosure. Those factors are readily recognized by one of ordinary skill in the art.

In general, the compounds of the present disclosure may be prepared by standard techniques known in the art and by known processes analogous thereto. For example, the compounds may be prepared according to methods described in U.S. Pat. No. 6,828,335, and U.S. application Ser. No. 13/375,878 filed on Dec. 2, 2011 which are incorporated by reference in its entirety. The present disclosure also encompasses indane acetic acid compounds and derivatives described in U.S. Pat. No. 7,112,597 in U.S. Pat. No. 8,541,618, and in U.S. Pat. No. 8,552,203, which are incorporated by references in their entirety. The present disclosure also encompasses indane acetic acid derivatives and their use described in US application publication number 2014/0086910, publication date Mar. 27, 2014 and in U.S. patent application Ser. No. 14/477,114, filed on Sep. 4, 2014, which are incorporated by references in their entirety.

### Evaluation of Biological Activity of Compounds

PPAR receptor agonist activity may be determined by conventional screening methods known to the skilled in the art. For example, methods described in U.S. Patent Application Publication No. 2007/0054907, 2008/0262047 and U.S. Pat. No. 7,314,879, which are incorporated by reference in their entireties.

The blood-brain barrier also eliminates lipophilic molecules by way of an active transport mechanism mediated by P-glycoprotein (P-gp). For a neurodegenerative disease therapeutic to be effective it has to achieve balance between passive diffusion in through the BBB, and active elimination out by the P-gp transporter, or other transportors. P-gp is an ATP-dependent, drug efflux pump for xenobiotic compounds with broad tissue distribution including the endothelia cells of the BBB. [Schinkel A H (April 1999). “P-Glycoprotein, a gatekeeper in the blood-brain barrier”. Advanced Drug Delivery Reviews 5 (36 (2-3)): 179-194.]. P-gp activity can be measured in pre-clinical in vitro studies.

### Animal Studies

The compounds described in the present disclosure may be tested in any animal model known to those skilled in the art. Exemplary animal models of Alzheimer' disease are listed on public web sites such as Alzforum (www.alzforum.com). Exemplary animal models for both Amyloid Related Imaging Anomolies (ARIA) and Cerebral Amyloid Angioplasty (CAA) include a variety of transgenic mouse models expressing the human Aβ precursor protein (APP). Many of these mouse models develop CAA in addition to senile plaques, whereas some of these models were generated specifically to study CAA. In addition, other animal models make use of a second stimulus, such as hypoperfusion or hyperhomocysteinemia (HHcy), to accelerate CAA.

Blood brain penetration studies can be carried out as described in recent publications and as is known by those skilled in the art. (See: Chang, K.L., et al. Influence of drug transporters and stereoselectivity on the brain penetration of pioglitazone as a potential medicine against Alzheimer's disease. Science Reports 2014, 5, 9000). In brief, A good measure of whether a small molecule penetrates the BBB and is not rapidly transported out, is the brain to plasma ratio of the drug. This is measured in pre-clinical animal models by determining plasma concentration vs time curves as in a standard pharmacokinetic study, and in addition harvesting brains and determining whole brain concentrations over time. The brain to plasma concentration ratio can them be determined at any time point, such as the Cₘₐₓ, or for the entire time curve (AUC, area under the curve). Clinically, FDG-PET can be used as a measure of the pharmacological effect of T3D-959, and an indirect measure of brain levels of drug.

For each model, the test result is compared with a control group that is not treated with the compounds described in the present disclosure. The treated animals are expected to demonstrate significant improvement in the performance of a variety of tests that measure steatosis, inflammation, fibrosis, dyslipidemia, and insulin resistance.

### Example 1

### Ethyl [(1S)-5-hydroxy-2,3-dihydro-1H-inden-1-yl]acetate

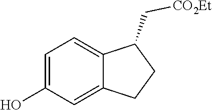 [SMILES: CCOC(=O)C[C@@H]1CCc2cc(O)ccc21]

Prepared in six steps from 5-methoxy indanone as described in US68283335.

### Example 2

### 2-[5-ethyl-2-(4-methoxyphenyl)-1,3-oxazol-4-yl]ethanol

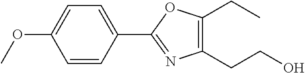 [SMILES: CCc1oc(-c2ccc(OC)cc2)nc1CCO]

Prepared from L-aspartic acid β-methyl ester hydrochloride, 4-methoxy benzoyl chloride and proprionic anhydride as generally described in US68283335.

### Example 3

### 2-[2-(4-methoxyphenyl)-5-methyl-1,3-oxazol-4-yl]ethanol

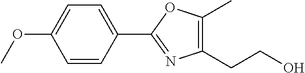 [SMILES: COc1ccc(-c2nc(CCO)c(C)o2)cc1]

Prepared from L-aspartic acid β-methyl ester hydrochloride, 4-methoxy benzoyl chloride and acetic anhydride as generally described in US68283335.

### Example 4

### 2-[5-Ethyl-2-(4-methylphenyl)-1,3-oxazol-4-yl]ethanol

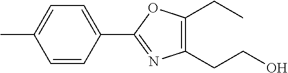 [SMILES: CCc1oc(-c2ccc(C)cc2)nc1CCO]

Prepared from L-aspartic acid β-methyl ester hydrochloride, p-toluoyl chloride and proprionic anhydride as generally described in US68283335.

### Example 5

### 2-[5-Methyl-2-(4-methylphenyl)-1,3-oxazol-4-yl]ethanol

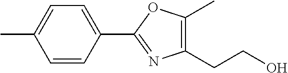 [SMILES: Cc1ccc(-c2nc(CCO)c(C)o2)cc1]

Prepared as from L-aspartic acid β-methyl ester hydrochloride, p-toluoyl chloride and acetic anhydride as described in US68283335.

### Example 6

### 2-[5-Ethyl-2-(4-ethylphenyl)-1,3-oxazol-4-yl]ethanol

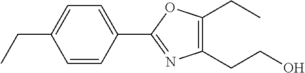 [SMILES: CCc1ccc(-c2nc(CCO)c(CC)o2)cc1]

Prepared from L-aspartic acid β-methyl ester hydrochloride, 4-ethyl benzoyl chloride and proprionic anhydride as generally described in US68283335.

### Example 7

### 2-[2-(4-Ethylphenyl)-5-methyl-1,3-oxazol-4-yl]ethanol

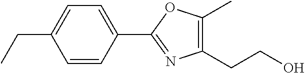 [SMILES: CCc1ccc(-c2nc(CCO)c(C)o2)cc1]

Prepared from L-aspartic acid β-methyl ester hydrochloride, 4-ethyl benzoyl chloride and acetic anhydride as generally described in US68283335.

### Example 8

### 2-(5-ethyl-2-(4-methoxyphenyl)oxazol-4-yl)ethyl benzenesulfonate

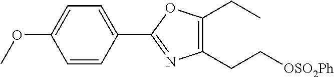

The intermediate from Example 2 (400.8 g), 15.0 g trimethylamine hydrochloride and 3.2 L dichloromethane was added to a 22 L reactor. The reaction mixture was stirred and cooled to 3.8° C. 680 mL of triethylamine was then added to the reactor. Benzenesulfonyl chloride (400 g) is slowly added to the reactor while maintaining the temperature below 12° C. The reaction was cooled to between 5° C. and 10° C. for three hours and then heated to 20° C. The contents of the reactor were stirred overnight at 24° C. Additional 3.2 L of dichloromethane was added to the reactor. The mixture was cooled to 5.0° C. and 205 mL 3-dimethylamino-1-propylamine was added. The mixture is stirred at 4.8° C. for 16 minutes. An aqueous citric acid solution (3 L of 1M) was slowly added to the reactor so as to maintain the temperature below 16° C. The resulting mixture was heated to 20° C. and stirred for 10 minutes. The phases were separated, and the organics were washed with 3 L of 1M citric acid solution, 3 L saturated sodium bicarbonate solution, 3 L brine solution, dried with magnesium sulfate, filtered and concentrated. The residue was treated with n-heptane and concentrated to give 542 g of crude 2-(5-ethyl-2-(4-methoxyphenyl)oxazol-4-yl)ethyl benzenesulfonate.

### Example 9

### (S)-Ethyl 2-(5-(2-(5-ethyl-2-(4-methoxyphenyl)oxazol-4-yl)ethoxy)-2,3-dihydro-1H-inden-1-yl)acetate

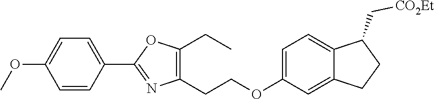 [SMILES: CCOC(=O)C[C@@H]1CCc2cc(OCCc3nc(-c4ccc(OC)cc4)oc3CC)ccc21]

A 22 L reactor was charged with 302.3 g of ethyl [(1S)-5-hydroxy-2,3-dihydro-1H-inden-1-yl]acetate (Example 1), 539.3 g crude 2-(5-ethyl-2-(4-methoxyphenyl)oxazol-4-yl)ethyl benzenesulfonate (Example 8) and 3.4 L acetonitrile. The mixture was stirred until all of the solids dissolved; then, 670.6 g cesium carbonate was added. The mixture is heated to 70° C. and held 16 hours. An additional charge of 60.2 g of compound from Example 1 was added to the reactor. The mixture was heated to 70° C. for one hour and additional cesium carbonate (316.9g) was added and heating was continued for 2.5 hours at 70° C. The reaction mixture was cooled to 24° C. and 4 L n-heptane, 2.4 L USP water, 2.4 L brine solution and 4 L ethyl acetate was charged to the reactor. The biphasic mixture was stirred for 5 minutes, then allowed to separate. The organic layer was washed with 2×2.4 L 5% sodium hydroxide solution and 2.4 L USP water, and 2.4 L brine. The solvent is removed via rotary evaporation until solids precipitate. Addition of 7.7 L n-heptane and stirring produced a slurry, which was filtered, and the filter cake was rinsed with the filtrate and then with 2.4 L n-heptane. The product air dried and then dried in a vacuum oven at 40° C. to give (S)-ethyl 2-(5-(2-(5-ethyl-2-(4-methoxyphenyl)oxazol-4-yl)ethoxy)-2,3-dihydro-1H-inden-1-yl)acetate as an off white solid.

### Example 10

### (S)-2-(5-(2-(5-ethyl-2-(4-methoxyphenyl)oxazol-4-yl)ethoxy)-2,3-dihydro-1H-inden-1-yl)acetic acid

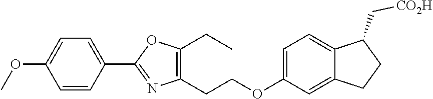 [SMILES: CCc1oc(-c2ccc(OC)cc2)nc1CCOc1ccc2c(c1)CC[C@H]2CC(=O)O]

A 22 L flask was charged with 478.9 g of (S)-ethyl 2-(5-(2-(5-ethyl-2-(4-methoxyphenyl)oxazol-4-yl)ethoxy)-2,3-dihydro-1H-inden-1-yl)acetate (Example 9) and 1.2 L ethanol and cooled to 20° C. To the 22 L flask was charged 1.6 L of 1N sodium hydroxide solution. The reaction mixture was heated to 65° C. for 30, then cooled to 25° C., and concentrated to an oil. A new reaction flask was charged with 4.8 L USP water and 1.9 L 1N hydrochloric acid solution, vigorously stirred and cooled to 23° C. The product oil was added to the solution via an addition funnel. The resulting suspension is stirred at approximately 23° C., and the pH is checked: 1.6 (target≤2). The solids were filtered and then washed with the mother liquor. The solids were washed with 3 L USP water and then with 1.9 L 1:1 ethanol SDA-2B: water. The filter cake was air dried for 4 hours and is then transferred to a vacuum oven. The solid wasdried under vacuum at 45° C. until a constant mass was achieved, producing (S)-2-(5-(2-(5-ethyl-2-(4-methoxyphenyl)oxazol-4-yl)ethoxy)-2,3-dihydro-1H-inden-1-yl)acetic acid as an off white solid.

### Example 11

### Sodium (S)-2-(5-(2-(5-ethyl-2-(4-methoxyphenyl)oxazol-4-yl)ethoxy)-2,3-dihydro-1H-inden-1-yl)acetate

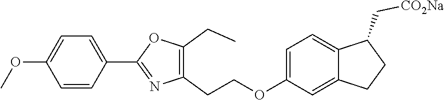

A 22 L reactor was charged with 3.8 L ethanol. Agitation was started, and the reactor was charged successively with 288.2 g sodium ethoxide solution (20.1% in ethanol) and with 378.4 g of (S)-2-(5-(2-(5-ethyl-2-(4-methoxyphenyl)oxazol-4-yl)ethoxy)-2,3-dihydro-1H-inden-1-yl)acetic acid (Example 10). The reaction mixture was heated to 40° C. for ˜20 minutes (until all solids are dissolved), and pH was checked (target pH 9-10).

The solution was filtered through a 10 micron filter membrane, returned to the reactor and heated to 40° C. The reactor was then charged with 3.4 L of filtered methyl t-butyl ether at such a rate that the temperature of the product solution is maintained at 40° C. throughout. The mixture is then seeded with 0.5 g Example 10 compound, and held at 42° C. for 40 minutes. An additional 3.4 L of filtered methyl t-butyl ether was added. The suspension was heated to 55° C. for 65 minutes. The suspension was cooled to 20-25° C. overnight then to 14° C. the next morning. The product was filtered under a nitrogen blanket, washed with 1.3 L filtered methyl t-butyl ether and dried to constant mass in a vacuum oven at 40° C. The bulk product was milled using a Comil with a 10 mesh sieve. The product is dried in a humidified environment at 40° C. NMR analysis showed ≤0.5% of ethanol by weight. Final product sodium (S)-2-(5-(2-(5-ethyl-2-(4-methoxyphenyl)oxazol-4-yl)ethoxy)-2,3-dihydro-1H-inden-1-yl)acetate was further dried at 45° C. under vacuum to obtain 306 g as a fine white solid.

### Example 12

### (S)-2-(5-(2-(2-(4-methoxyphenyl)-5-methyloxazol-4-yl)ethoxy)-2,3-dihydro-1H-inden-1-yl)acetic acid

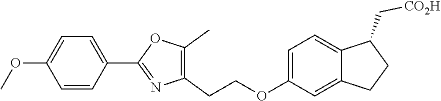 [SMILES: COc1ccc(-c2nc(CCOc3ccc4c(c3)CC[C@H]4CC(=O)O)c(C)o2)cc1]

(S)-Ethyl 2-(5-hydroxy-2,3-dihydro-1H-inden-1-yl)acetate from Example 1 and 2-(2-(4-methoxyphenyl)-5-methyloxazol-4-yl)ethanol from Example 3 were combined and reacted as in Examples 8,9 and 10 to give (S)-2-(5-(2-(2-(4-methoxyphenyl)-5-methyloxazol-4-yl)ethoxy)-2,3-dihydro-1H-inden-1-yl)acetic acid as an off white solid.

### Example 13

### (S)-2-(5-(2-(5-ethyl-2-p-tolyloxazol-4-yl)ethoxy)-2,3-dihydro-1H-inden-1-yl)acetic acid

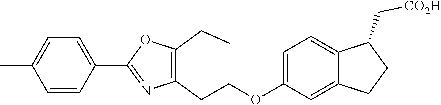 [SMILES: CCc1oc(-c2ccc(C)cc2)nc1CCOc1ccc2c(c1)CC[C@H]2CC(=O)O]

(S)-Ethyl 2-(5-hydroxy-2,3-dihydro-1H-inden-1-yl)acetate from Example 1 and 2-(5-ethyl-2-p-tolyloxazol-4-yl)ethanol from Example 4 were combined and reacted as in Examples 8, 9 and 10 to give (S)-2-(5-(2-(5-ethyl-2-p-tolyloxazol-4-yl)ethoxy)-2,3-dihydro-1H-inden-1-yl)acetic acid as an off white solid.

### Example 14

### (S)-2-(5-(2-(5-methyl-2-p-tolyloxazol-4-yl)ethoxy)-2,3-dihydro-1H-inden-1-yl)acetic acid

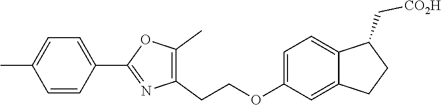 [SMILES: Cc1ccc(-c2nc(CCOc3ccc4c(c3)CC[C@H]4CC(=O)O)c(C)o2)cc1]

(S)-Ethyl 2-(5-hydroxy-2,3-dihydro-1H-inden-1-yl)acetate from Example 1 and 2-(5-methyl-2-p-tolyloxazol-4-yl)ethanol from Example 5 were combined and reacted as described in Examples 8, 9 and 10 to give (S)-2-(5-(2-(5-methyl-2-p-tolyloxazol-4-yl)ethoxy)-2,3-dihydro-1H-inden-1-yl)acetic acid as an off white solid.

### Example 15

### (S)-2-(5-(2-(5-ethyl-2-(4-ethylphenyl)oxazol-4-yl)ethoxy)-2,3-dihydro-1H-inden-1-yl)acetic acid

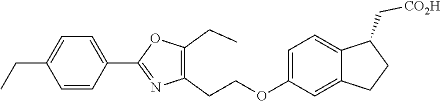 [SMILES: CCc1ccc(-c2nc(CCOc3ccc4c(c3)CC[C@H]4CC(=O)O)c(CC)o2)cc1]

(S)-Ethyl 2-(5-hydroxy-2,3-dihydro-1H-inden-1-yl)acetate from Example 1 and 2-(5-ethyl-2-(4-ethylphenyl)oxazol-4-yl)ethanol from Example 6 were combined and reacted as described in Examples 8, 9 and 10 to give (S)-2-(5-(2-(5-ethyl-2-(4-ethylphenyl)oxazol-4-yl)ethoxy)-2,3-dihydro-1H-inden-1-yl)acetic acid as an off white solid.

### Example 16

### (S)-2-(5-(2-(5-ethyl-2-(4-ethylphenyl)oxazol-4-yl)ethoxy)-2,3-dihydro-1H-inden-1-yl)acetic acid

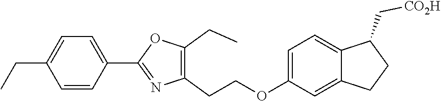 [SMILES: CCc1ccc(-c2nc(CCOc3ccc4c(c3)CC[C@H]4CC(=O)O)c(CC)o2)cc1]

(S)-Ethyl 2-(5-hydroxy-2,3-dihydro-1H-inden-1-yl)acetate from Example 1 and 2-(2-(4-ethylphenyl)-5-methyloxazol-4-yl)ethanol from Example 7 were combined and reacted as described in Examples 8, 9 and 10 to give (S)-2-(5-(2-(2-(4-ethylphenyl)-5-methyloxazol-4-yl)ethoxy)-2,3-dihydro-1H-inden-1-yl)acetic acid as an off white solid.

### Example 17

### Human PPAR Activation by Compound of Example 11 in Transient Transfection Study

The table summarize the results of studies performed in transfected CV-1 cells with three different lots of Compound of Example 11. These results showed an average EC₅₀ for activation of the human gamma subtype of 297 nM with a 74% maximal response. In similar experiments rosiglitazone had a human gamma subtype EC₅₀ of 130 nM. The average EC₅₀ for activation of the human δ subtype was 19 nM with a 76% maximal response. In similar experiments GW501516 had a human δ subtype EC₅₀ of 1.3 nM. The average EC₅₀ for activation of human alpha subtype was 530 nM with a significantly reduced maximal response. These results demonstrate that (S)-2-(5-(2-(5-ethyl-2-(4-methoxyphenyl)oxazol-4-yl)ethoxy)-2,3-dihydro-1H-inden-1-yl)acetate is a potent, selective agonist of the PPARO and PPARy subtypes, with a 15-fold greater potency in activating the human PPARo subtype over the human PPARγ subtype and about a 30-fold selectivity over the human PPAR +balpha subtype.

Summary of Human PPAR Activation by Compound of Example 11
in Transient Transfection Studies

| | Material | Use | PPARγ | PPARδ | PPARα |
|---|---|---|---|---|---|
| EC₅₀ (nM) | Lot A | Non GMP | 220 | 15 | 488 |
| | Lot B | GLP Nonclinical | 270 | 27 | 750 |
| | | Studies | | | |
| | Lot C | cGMP material | 400 | 16 | 360 |

| | | | |
|---|---|---|---|
| Average EC₅₀ (nM) | 297 | 19 | 530 |
| Average % maximal | 74 | 76 | 49 |
| response | | | |
| Peak effect | 29.49 | 17.68 | 1.87 |

### Example 18

### Effect of P-gp Inhibitor Verapamil on Caco-2 Permeability of Compound of Example 11

The human Caco-2 permeability of Compound of Example 11 was evaluated. High permeability of (S)-2-(5-(2-(5-ethyl-2-(4-methoxyphenyl)oxazol-4-yl)ethoxy)-2,3-dihydro-1H-inden-1-yl)acetate was observed in the absence of a P-glycoprotein (P-gp) inhibitor (Papparent=1144 nm/sec); no significant change in permeability was observed in the presence of the P-gp inhibitor (verapamil). These results indicate that (S)-2-(5-(2-(5-ethyl-2-(4-methoxyphenyl)oxazol-4-yl)ethoxy)-2,3-dihydro-1H-inden-1-yl)acetate is not a substrate for P-gp.

### Example 19

### Determination of Brain to Plasma Ratios for Compound of Example 11

Over 98% of drugs in clinical development for all diseases fail to adequately penetrate the blood brain barrier (BBB) to provide adequate brain exposure. For compounds of the present disclosure to be effective in treating neurotoxicity or neurodegeneration, it must have an ability to cross the BBB and penetrate the brain. To assess the ability of compounds of the present disclosure to cross the BBB, the pharmacokinetics and brain-to-plasma ratio of compound of Example 11, (Sodium (S)-2-(5-(2-(5-ethyl-2-(4-methoxyphenyl)oxazol-4-yl)ethoxy)-2,3-dihydro-1H-inden-1-yl)acetate was evaluated after oral dosing in male Sprague-Dawley rats. The test compound was dosed at 3 mg/kg from normal saline. Plasma and brain levels were determined by LC-MS/MS at pre-determined time points. Pharmacokinetic parameters were estimated by a non-compartmental model using WinNonlin v5.3 software. After oral dosing of test compound at 3 mg/kg, plasma Cₘₐₓ values of 1547±248 ng/mL were reached at 5 hours post dose. The average plasma half-life was 3.33 hours. The plasma exposure as measured by AUCₗₐₛₜ was 9569±1190 hr*ng/ml. Brain/Plasma ratios were found to be 0.361±0.142, 0.220±0.033, 0.171±0.011, 0.328±0.154, and 0.350±0.077 at 1, 3, 5, 7, and 12 hours, respectively. Results from the 15 rat experiment are shown in Table 2.

TABLE 2
Rat Brain Penetrance Pharmacokinetics
of Compound of Example 11

| Time | | Plasma | | | | |
|---|---|---|---|---|---|---|
| point | | Conc. | Brain Tissue | B/P | Average | |
| (br) | Rat# | (ng/mL) | Cone. (ng/g) | Ratio | (ng/g) | SD |
| 1.0 | 840 | 453 | 161 | 0.36 | 0.361 | 0.142 |
| | 841 | 199 | 101 | 0.51 | | |
| | 842 | 1020 | 226 | 0.22 | | |
| 3.0 | 843 | 910 | 234 | 0.26 | 0.220 | 0.033 |
| | 844 | 1270 | 269 | 0.21 | | |
| | 845 | 1430 | 274 | 0.19 | | |
| 5.0 | 846 | 1260 | 208 | 0.17 | 0.171 | 0.011 |
| | 847 | 1340 | 247 | 0.18 | | |
| | 848 | 2040 | 333 | 0.16 | | |
| 7.0 | 849 | 1240 | 203 | 0.16 | 0.328 | 0.154 |
| | 850 | 368 | 130 | 0.35 | | |
| | 851 | 462 | 216 | 0.47 | | |
| 12.0 | 852 | 345 | 104 | 0.30 | 0.350 | 0.077 |
| | 853 | 259 | 114 | 0.44 | | |
| | 854 | 377 | 117 | 0.31 | | |

### Example 20

### FDG-PET Data from Clinical Study with Compound of Example 11

A total of 36 subjects were enrolled into a two week long treatment study: 9 subjects randomized into 3 mg and 10 mg, 10 subjects randomized into 30 mg and 8 subjects randomized into 90 mg. The average age was 75 years with more than half of the subjects ranging between ages 65 to 84. Males and females were equally represented across all dose levels. The majority were non-Hispanic and of white origin. One half of the enrolled subjects carried one or two copies of the E4 allele for APOE genotype. FDG-PET scans were obtained at three clinical sites as described in the T3D959-201 Clinical Protocol. PET scans were obtained at baseline (BL) and again at end of treatment (EOT) for patients in all four dosage groups of Compound of Example 11 (3 mg, 10 mg, 30 mg, and 90 mg). The imaging protocol developed for the AD Neuroimaging Initiative (ADNI2) was used to collect the data. Overnight fasted subjects (blood glucose<180 mg/dL) received IV injection of 5mCi [F18] fluoro-deoxyglucose as a single bolus. Subjects were instructed to lay supine with eyes open and forward. Thirty minutes after dosing, six 5-minute (total 30-min) emission scans were acquired. FDG-PET measurements obtained are relative to two reference regions, Whole Brain (WB) and cerebral White Matter (WM), for the computation of the changes in Relative Cerebral Metabolic Rate for glucose over the dosing period or: ΔR CMRgl (EOT-BL). The key primary outcome, was a global index, (sROI index) calculated from the average bq/voxel reading over an empirically pre-specified statistical Region of Interest (sROI), known to be affected by AD, which is normalized by the average bq/voxel for an empirically pre-specifed statistic ROI that is relatively spared. Change in the sROI index from BL to EOT is reported as ΔsROI. A second outcome was the determination of ΔR CMRgl (EOT-BL) for four pre-specified known AD-affected regions of interest (ROIs): 1) Posterior Cingulate (PC), 2) Precuneus (PreC), 3) Bilateral Middle Temporal Gyrus (BMTG), and 4) Right Inferior Parietal Lobule (RIPL). The final main outcome was a exploratory voxel-wise analysis of the whole brain to identify Regions of Statistically Significant Differences (ROSD) for ΔR CMRgl (EOT-BL) with uncorrected p<0.005. The voxel-wise analysis results are presented as slice by slice statistical map display superimposed on the anatomical T1 MR images with ROSDs highlighted in yellow. The R CMRgl values referred to in this report are calculated as the ratio of the average of the bq/voxel reading for each voxel, over each ROI, and divided by the average bq/voxel over the reference region used, e.g., WB or WM.

Results from the study suggest the following:

Compound of Example 11 (T3D-959) penetrates the Blood Brain Barrier (BBB) even at the lowest 3 mg. This conclusion comes from the voxel-wise (SPM) analysis and assumes that the findings are not solely attributable to small number of subjects, or the short test-retest interval.

T3D-959 altered the glucose metabolism in the brain. Increases and decreases in relative regional glucose metabolism (ΔR CMRgl (EOT-BL)) were observed over the treatment period. Table 4 below shows multiple regions of the brain with positive ΔR CMRgl (EOT-BL) Relative to Average Whole Brain for the 90 mg dose group. These are regions which are responding better to T3D-959 than the average whole brain.

Brain Regions with Positive Δ R CMRgI (EOT-BL) Relative to
Average Whole Brain for the 90 mg Group (ANOVA design)

| | | Talairach |
|---|---|---|
| | | Coordinates |
| Brain Region | P value | (x, y, z) |
| Putamen_R* | 6.23E−07 | 32 −13 8 |
| Vermis_3 | 3.14E−06 | −2 −35 −5 |
| Putamen_R | 3.15E−06 | 32 −11 4 |
| ParaHippocampal_R | 5.05E−06 | 24 −5 −20 |
| Putamen_R | 7.42E−06 | 32 −11 12 |
| Caudate_R | 3.08E−05 | 10 7 −10 |
| Hippocampus_L | 3.58E−05 | −26 −9 −20 |
| Fusiform_R | 3.79E−05 | 30 −2 −30 |
| Cingulum_Mid_L | 1.26E−04 | −14 −18 38 |
| Frontal_Mid_L | 1.62E−04 | −24 13 25 |
| Fusiform_R | 2.47E−04 | 30 −2 −34 |
| Insula_R | 2.52E−04 | 42 −14 −6 |
| Temporal_Inf_R | 9.54E−04 | 30 2 −40 |

*Survived multiple comparisons FWE = 0.050
Total number of voxels which survived uncorrected p = 0.001 is 2136

Demonstration of the activity of the compounds of the present disclosure may be accomplished through in vitro, ex vivo and in vivo assays that are well known in the art. The effect of T3D-959 on the FDG-PET outcomes appears to be T3D-959 dose-dependent, with larger effects observed at larger doses. This observation comes from the sROI, and anatomical ROI analyses as well as the exploratory voxel-wise SPM analysis. The image displays below from the voxel-wise analysios shows a clear increase in the spatial extent of the regions of the yellow regions from 70 voxels to 2136 voxels as the dose increases from 10 mg to 90 mg. The yellow regions are made up of voxels with statistically significant differences from baseline to end of treatment (ROSDs).

### ROSDs with Positive ΔR CMRgl (EOT-BL) Relative to Whole Brain (p<0.005) for with Increasing Doses of T3D-959

Those skilled in the art to which the present disclosure pertains may make modifications resulting in other embodiments employing principles of the present disclosure without departing from its spirit or characteristics, particularly upon considering the foregoing teachings. Accordingly, the described embodiments are to be considered in all respects only as illustrative, and not restrictive, and the scope of the present disclosure is, therefore, indicated by the appended claims rather than by the foregoing description or drawings. Consequently, while the present disclosure has been described with reference to particular embodiments, modifications of structure, sequence, materials and the like apparent to those skilled in the art still fall within the scope of the disclosure as claimed by the applicant.

### Example 21

### FDG-PET Study of the Visual Cortex

### AD Hoc Analysis of Visual Cortex and Sensory Motor Cortex

Ad Hoc analysis of two additional brain regions, the left and right visual cortex and the left and right sensory motor cortex, provided ΔR CMRgl values with both WB and WM as the RRs. The Banner Alzheimer's Institue provided the data, and AppStat did the calculations.

The results are summarized in Tables 3 and 4 below. This analysis was meant to answer the question would these two brain regions, which are not affected by AD, respond more like the AD affected Precuneus, with negative, ΔR CMRgl (EOT-BL) values or the AD spared Putamen with which has positive ΔR CMRgl (EOT-BL) values at the highest doses.

The results in Table 3 show that the ΔR CMRgl (EOT-BL) values for both the left and right Sensory Motor Cortex, with WM as the RR, are small for all doses. The small ΔR CMRgl (EOT-BL) values suggests the visual cortex responds similarly increasing doses of T3D-959 as WM, and thus is not like Precuneus, but perhaps more like the Putamen.

The left and right Visual Cortex, on the other hand, responds to increasing doses of T3D-959 more like the four prespecified AD affected regions, including the Precuneus, in the original study. The data in the table below shows that for the 3 and 10 mg doses, ΔR CMRgl (EOT-BL) values are small, suggesting they respond similarly to WM. However, at 30 and 90 mg, ΔR CMRgl (EOT-BL) values are negative, and exceed a standard deviation, suggesting that the Visual Cortex does not respond as well to these two high doses of T3D-959 as WM. This is similar to how the Precuneus responds relative to WM (see data above).

TABLE 3
Visual and Sensory Motor Cortex Δ R CMRgI (EOT-BL)
Values Relative to White Matter
White Matter as Reference Region

| Dose | Visual Cortex | | Sensory Motor Cortex | |
|---|---|---|---|---|
| 3 mg | Left | Right | Left | Right |
| Ave | −0.0219 | −0.0272 | −0.0005 | −0.0151 |
| STD | 0.0906 | 0.0965 | 0.0287 | 0.0478 |
| 10 mg | | | | |
| Ave | −0.0014 | −0.0033 | 0.0005 | −0.0005 |
| STD | 0.0508 | 0.0507 | 0.0329 | 0.0177 |
| 30 mg | | | | |
| Ave | −0.0314 | −0.0408 | −0.0040 | −0.0229 |
| STD | 0.0274 | 0.0254 | 0.0220 | 0.0342 |
| 90 mg | | | | |
| Ave | −0.0775 | −0.0730 | −0.0076 | −0.0078 |
| STD | 0.0535 | 0.0556 | 0.0532 | 0.0518 |

The results from a corresponding analysis with Whole Brain as the reference region are shown in the table below. The ΔR CMRgl (EOT-BL) values for the left and right Visual cortex and the left and right Sensory Motor cortex, for the first three doses are small, suggesting that Visual Cortex and Sensory Motor Cortex respond similarly to 3, 10 and 30 mg doses of T3D-959 as the average whole brain. At the 90 mg dose, the ΔR CMRgl (EOT-BL) values for the left and right Visual cortex are negative, and close to a standard deviation, suggesting that at this high dose, perhaps the visual cortex does not respond as well to drug as average WB. Conversely, the Sensory Motor Cortex ΔR CMRgl (EOT-BL) values are positive, but like the three higher doses, are well within a SD, suggesting it responds to T3D-959 similarly to the average Whole Brain, across all four doses.

TABLE 10
Visual and Sensory Motor Cortex Δ R CMRgI (EOT-BL)
Values Relative to average Whole Brain
Whole Brain as Reference Region

| Dose | Visual Cortex | | Sensory Motor Cortex | |
|---|---|---|---|---|
| 3 mg | Left | Right | Left | Right |
| Ave | −0.0196 | −0.0269 | 0.0152 | −0.0037 |
| STD | 0.0894 | 0.0970 | 0.0684 | 0.0664 |
| 10 mg | | | | |
| Ave | −0.0032 | −0.0053 | 0.0008 | 0.0003 |
| STD | 0.0463 | 0.0505 | 0.0355 | 0.0383 |
| 30 mg | | | | |
| Ave | −0.0158 | −0.0271 | 0.0213 | −0.0040 |
| STD | 0.0317 | 0.0375 | 0.0457 | 0.0603 |
| 90 mg | | | | |
| Ave | −0.0685 | −0.0604 | 0.0244 | 0.0251 |
| STD | 0.0536 | 0.0607 | 0.0826 | 0.0852 |

### Example 22

### BOLD fMRI Data from Clinical Study with Compound of Example 11

A total of 36 subjects were enrolled into a two week long treatment study: 9 subjects randomized into 3 mg and 10 mg, 10 subjects randomized into 30 mg and 8 subjects randomized into 90 mg. The average age was 75 years with more than half of the subjects ranging between ages 65 to 84. Males and females were equally represented across all dose levels. The majority were non-Hispanic and of white origin. One half of the enrolled subjects carried one or two copies of the E4 allele for APOE genotype. BOLD fMRI scans were obtained at three clinical sites as described in the T3D959-201 Clinical Protocol. BOLD fMRI scans were obtained at baseline (BL) and again at end of treatment (EOT) for patients in all four dosage groups of Compound of Example 11 (3 mg, 10 mg, 30 mg, and 90 mg).

T3D Therapeutics completed an exploratory analysis of the Phase 2A non-placebo-controlled feasibility study to assess the therapeutic effects of T3D959 in patients with mild-to-moderate Alzheimer's disease (AD) via task-free functional magnetic resonance imaging (tf-fMRI). A total of 34 subjects participated and were split into 4 groups that took different amounts of T3D959 over the course of the study: 3 mg, 10 mg, 30 mg, and 90 mg. A baseline tf-fMRI scan was obtained prior to treatment and another two weeks after. Sixteen tf-fMRI metrics were calculated at each time point and summarized for each patient group to assess the therapeutic effect of T3D959.

The T3D clinical trial investigated the following metrics:
1. Goodness of Fit of the subject's default mode network regions compared to those of an elderly control group.
2. The correlation strength between the hippocampus and precuneus.
3. The global efficiency calculated from 11 pre-defined default mode network regions.
4. The global efficiency calculated from 90 pre-defined cerebral regions based on the automated anatomical labeling atlas.
5. The amplitude of low frequency fluctuations (ALFF) and ratio amplitude of low frequency fluctuations (fALFF) from the right posterior cingulate cortex (PCC) and precuneus (PreC) in the low frequency oscillations (LFO) range (0.01-0.08 Hz).
6. The ALFF and fALFF from the left PCC and PreC in the LFO range.
7. The regional homogeneity (ReHo) in the right PCC and PreC.
8. The ReHo in the left PCC and PreC.
9. The ALFF and fALFF from the right inferior parietal lobule (IPL) in the LFO range.
10. The ALFF and fALFF from the left IPL in the LFO range.
11. The ReHo in the right IPL
12. The ReHo in the left IPL.

Results from the ALFF, fALFF and ReHo analyses are shown in the Table 5 below.

TABLE 5
Change from Baseline EOT BOLD fMRI

| Imaging Variable | | 3 mg | 10 mg | 30 mg | 90 mg |
|---|---|---|---|---|---|
| Visit Windows | Statistics | (N = 9) | (N = 9) | (N = 10) | (N = 8) |
| fALFF-IPPC-PreC | N | 8 | 9 | 9 | 8 |
| | Mean | 0.0121 | 0.0068 | −0.0132 | −0.0167 |
| | (SD) | (0.03246) | (0.03510) | (0.03955) | (0.03284) |
| | Median | 0.0019 | −0.0068 | −0.0141 | −0.0166 |
| fALFF-rPPC-PreC | N | 8 | 9 | 9 | 8 |
| | Mean | −0.0080 | 0.0084 | −0.0007 | −0.0118 |
| | (SD) | (0.04094) | (0.04325) | (0.03913) | (0.04236) |
| | Median | −0.0229 | −0.0028 | −0.0133 | 0.0039 |
| ReHo-IPPC-PreC | N | 8 | 9 | 9 | 8 |
| | Mean | −0.0665 | 0.0237 | −0.0369 | 0.0194 |
| | (SD) | (0.18502) | (0.14028) | (0.09702) | (0.20061) |
| | Median | −0.0428 | 0.0523 | −0.0398 | 0.0566 |
| ReHo-rPPC-Prec | N | 8 | 9 | 9 | 8 |
| | Mean | −0.0390 | −0.0663 | −0.0895 | −0.0041 |
| | (SD) | (0.06406) | (0.22082) | (0.10541) | (0.15836) |
| | Median | −0.0081 | 0.0153 | −0.1176 | 0.0177 |
| ALFF-IIPL | N | 8 | 9 | 9 | 8 |
| | Mean | −0.0635 | 0.0104 | −0.0102 | −0.0401 |
| | (SD) | (0.09445) | (0.05763) | (0.12016) | (0.05657) |
| | Median | −0.0393 | 0.0273 | −0.0031 | −0.0560 |
| ALFF-rIPL | N | 8 | 9 | 9 | 8 |
| | Mean | −0.0493 | −0.0276 | 0.0346 | −0.1089 |
| | (SD) | (0.11288) | (0.12720) | (0.09654) | (0.13935) |
| | Median | −0.0587 | −0.0278 | 0.0332 | −0.0671 |
| fALFF-IIPL | N | 8 | 9 | 9 | 8 |
| | Mean | −0.0296 | 0.0164 | −0.0107 | −0.0350 |
| | (SD) | (0.03988) | (0.02269) | (0.06563) | (0.04570) |
| | Median | −0.0311 | 0.0097 | −0.0034 | −0.0211 |
| fALFF-rIPL | N | 8 | 9 | 9 | 8 |
| | Mean | −0.0342 | 0.0061 | 0.0023 | −0.0395 |
| | (SD) | (0.03912) | (0.03286) | (0.02771) | (0.05327) |
| | Median | −0.0327 | 0.0096 | 0.0001 | −0.0313 |
| ReHo-IIPL | N | 8 | 9 | 9 | 8 |
| | Mean | −0.0694 | 0.0147 | −0.0249 | −0.0062 |
| | (SD) | (0.15420) | (0.14620) | (0.19178) | (0.06575) |
| | Median | −0.0822 | −0.0201 | −0.0135 | −0.0121 |
| ReHo-rIPL | N | 8 | 9 | 9 | 8 |
| | Mean | −0.0694 | 0.0147 | −0.0249 | −0.0062 |
| | (SD) | (0.15420) | (0.14620) | (0.19178) | (0.06575) |
| | Median | −0.0822 | −0.0201 | −0.0135 | −0.0121 |

### Example 23

### Re-Analysis of BOLD fMRI Data from Clinical Study with Compound of Example 11

A re-analysis of the underlying fMRI data and results will be performed.

1. A method of treating an amyloid-related brain disorder comprising Alzheimer's disease or amyloid-related imaging abnormality (ARIA), the method comprising administrating a therapeutically effective amount of a compound of Formula I and an anti-amyloid beta antibody: 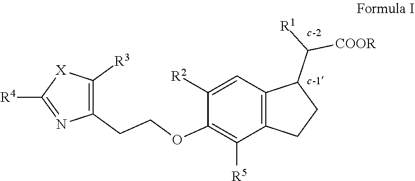
wherein
R is H, Na⁺, Li⁺, Ca⁺, K⁺, N⁺(C₁₋₆)₄, or C₁₋₆ alkyl;
R¹ is H, C₁₋₆ alkyl, C₃₋₆ cycloalkyl, or C₂₋₆ alkenyl, or C₁₋₆ alkoxy, each of which may be unsubstituted or substituted with fluoro, or phenyl which may be unsubstituted or substituted with R⁶, “c-2” is defined as the second carbon of the acetic acid portion of Formula I, and “c-1′” is the first carbon of the indane group of Formula I;
R² is H, halo, or C₁₋₆ alkyl which may be unsubstituted or substituted with C₁₋₆ alkoxy, oxo, fluoro, or
R² is phenyl, furyl, thienyl, pyrrolyl, oxazolyl, thiazolyl, imidazolyl, pyrazolyl, pyridyl, pyrrolidinyl, piperidinyl, tetrahydropyranyl, piperazinyl, or morpholinyl, each of which may be unsubstituted or substituted with R⁶;
R³ is H, C₁₋₆ alkyl, or phenyl, which may be unsubstituted or substituted with R⁶;
X is O or S;
R⁴ is phenyl, naphthyl, furyl, thienyl, pyrrolyl, oxazolyl, thiazolyl, pyrazolyl, pyridyl, pyrimidinyl, benzofuryl, benzothienyl, indolyl, indazolyl, benzoxazolyl, benzimidazolyl, quinolyl, isoquinolyl, or 1,4-benzodioxanyl, each of which may be unsubstituted or singlularly or multiply substituted with R⁶, or with phenyl, furyl, thienyl, pyrrolyl, each of which may be unsubstituted or singularly or multiply substituted with R⁶; or
R⁴ is C₁-C₆ alkyl or C₃-C₈ cycloalkyl, either of which may be unsubstituted or substituted with fluoro, oxo, or C₁-C₆ alkoxy which may be unsubstituted or substituted with C₁-C₆ alkoxy, or phenyl optionally substituted with R⁶, each of which may be substituted with phenyl, naphthyl, furyl, thienyl, pyrrolyl, each of which may be unsubstituted or further substituted with R⁶, or any C₁-C₆ alkyl may also be substituted with C₃-C₈ cycloalkyl or with phenoxy which may be unsubstituted or substituted with R⁶ or with phenyl, furyl, thienyl, pyrrolyl, each of which may be unsubstituted or substituted with R⁶, or
R⁵ is H, halo or C₁₋₆ alkyl optionally substituted with oxo;
R⁶ is halo, CF₃, C₁₋₆ alkyl optionally substituted with oxo or hydroxy, or C₁₋₆ alkoxy optionally substituted with fluoro;
wherein R³ may be attached to the heterocyclic moiety of the compound of Formula I at either the 4 or 5 position, and, accordingly, the remaining portion of the molecule will be attached at the remaining available carbon atom; or
a pharmaceutically acceptable salt, stereoisomer, enantiomer, racemate or combination thereof.

2. The method of claim 1, wherein the anti-amyloid beta antibody comprises aducanumab, bapineuzumab, crenezumab, gantenerumab, or solanezumab.

3. The method of claim 1, wherein
R¹ is H;
R² is H, halo, or C₁₋₆ alkyl which may be unsubstituted or substituted with C₁₋₆ alkoxy, oxo, fluoro;
R⁴ is phenyl, which may be unsubstituted or singularly or multiply substituted with R⁶; and
R⁵ is H, halo or C₁-C₆ alkyl optionally substituted with C₁-C₆ alkoxy, oxo, fluoro.

4. The method of claim 1, wherein
R¹ is H;
R² is H or halo;
R³ is H or C₁₋₆ alkyl;
R⁴ is phenyl, which may be singularly or multiply substituted with R⁶;
R⁵ is H or halo;
R⁶ is halo, CF₃, C₁₋₆ alkyl or C₁₋₆ alkoxy; and
c-1′ has the S stereochemistry.

5. The method of claim 1, wherein
R¹ is H;
R² is H or halo;
R³ is C₁₋₆ alkyl;
X is O;
R⁴ is phenyl, which may be singularly or multiply substituted with R⁶;
R⁵ is H or halo;
R⁶ is halo, CF₃, C₁₋₆ alkyl or C₁₋₆ alkoxy; and
c-1′ has the S stereochemistry.

6. The method of claim 1, wherein
R¹ is H;
R² is F;
R³ is C₁₋₆ alkyl;
X is O;
R⁴ is phenyl, which may be singularly or multiply substituted with R⁶;
R⁵ is F;
R⁶ is halo, CF₃, C₁₋₆ alkyl, C₁₋₆ alkoxy or C₁₋₆ alkyl; and
c-1′ has the S stereochemistry.

7. The method of claim 1, wherein
R is H or Na;
R¹ is H;
R² is H;
R³ is C₁₋₆ alkyl;
X is O;
R⁴ is phenyl, which may be singularly or multiply substituted with R⁶;
R⁵ is H;
R⁶ is halo, CF₃, C₁₋₆ alkyl, C₁₋₆ alkoxy or C₁₋₆ alkyl; and
c-1′ has the S stereochemistry.

8. The method of claim 1, wherein
R is H or Na;
R¹ is H;
R² is H;
R³ is C₁₋₆ alkyl;
X is S;
R⁴ is phenyl, which may be singularly or multiply substituted with R⁶;
R⁵ is H;
R⁶ is halo, CF₃, C₁₋₆ alkyl, C₁₋₆ alkoxy or C₁₋₆ alkyl; and
c-1′ has the S stereochemistry.

9. The method of claim 1, wherein compound is selected from the group consisting of

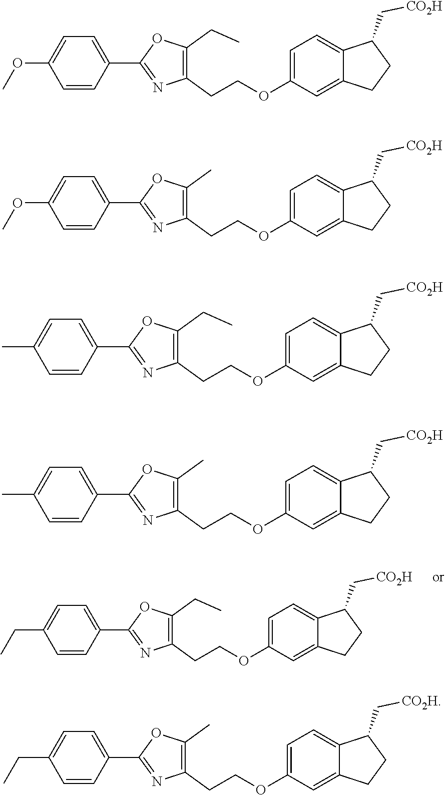 [SMILES: CCc1oc(-c2ccc(OC)cc2)nc1CCOc1ccc2c(c1)CC[C@H]2CC(=O)O ; COc1ccc(-c2nc(CCOc3ccc4c(c3)CC[C@H]4CC(=O)O)c(C)o2)cc1 ; CCc1oc(-c2ccc(C)cc2)nc1CCOc1ccc2c(c1)CC[C@H]2CC(=O)O ; Cc1ccc(-c2nc(CCOc3ccc4c(c3)CC[C@H]4CC(=O)O)c(C)o2)cc1 ; CCc1ccc(-c2nc(CCOc3ccc4c(c3)CC[C@H]4CC(=O)O)c(CC)o2)cc1 ; CCc1ccc(-c2nc(CCOc3ccc4c(c3)CC[C@H]4CC(=O)O)c(C)o2)cc1]

10. A method of reducing an amyloid-related brain disorder such as Alzheimer's disease or an amyloid-related imaging abnormality (ARIA), the method comprising administrating a therapeutically effective amount of: 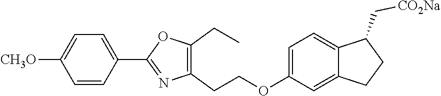
or
a pharmaceutically acceptable salt thereof and an anti-amyloid beta antibody, wherein the anti-amyloid beta antibody comprises aducanumab, bapineuzumab, crenezumab, gantenerumab, or solanezumab.

11. A pharmaceutical composition comprising at least one pharmaceutically acceptable excipient and a therapeutically effective amount of the compound of claim 1.

12. A method of treating a vasogenic edema comprising administrating a therapeutically effective amount of a compound of Formula I: 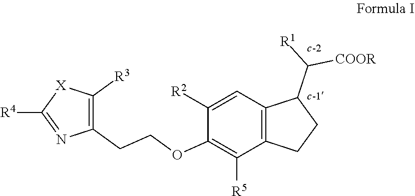
wherein
R is H, Na⁺, Li⁺, Ca⁺, K⁺, N⁺(C₁₋₆)₄, or C₁₋₆ alkyl;
R¹ is H, C₁₋₆ alkyl, C₃₋₆ cycloalkyl, or C₂₋₆ alkenyl, or C₁₋₆ alkoxy, each of which may be unsubstituted or substituted with fluoro, or phenyl which may be unsubstituted or substituted with R⁶, “c-2” is defined as the second carbon of the acetic acid portion of Formula I, and “c-1′” is the first carbon of the indane group of Formula I;
R² is H, halo, or C₁₋₆ alkyl which may be unsubstituted or substituted with C₁₋₆ alkoxy, oxo, fluoro, or
R² is phenyl, furyl, thienyl, pyrrolyl, oxazolyl, thiazolyl, imidazolyl, pyrazolyl, pyridyl, pyrrolidinyl, piperidinyl, tetrahydropyranyl, piperazinyl, or morpholinyl, each of which may be unsubstituted or substituted with R⁶;
R³ is H, C₁₋₆ alkyl, or phenyl, which may be unsubstituted or substituted with R⁶;
X is O or S;
R⁴ is phenyl, naphthyl, furyl, thienyl, pyrrolyl, oxazolyl, thiazolyl, pyrazolyl, pyridyl, pyrimidinyl, benzofuryl, benzothienyl, indolyl, indazolyl, benzoxazolyl, benzimidazolyl, quinolyl, isoquinolyl, or 1,4-benzodioxanyl, each of which may be unsubstituted or singlularly or multiply substituted with R⁶, or with phenyl, furyl, thienyl, pyrrolyl, each of which may be unsubstituted or singularly or multiply substituted with R⁶; or
R⁴ is C₁-C₆ alkyl or C₃-C₈ cycloalkyl, either of which may be unsubstituted or substituted with fluoro, oxo, or C₁-C₆ alkoxy which may be unsubstituted or substituted with C₁-C₆ alkoxy, or phenyl optionally substituted with R⁶, each of which may be substituted with phenyl, naphthyl, furyl, thienyl, pyrrolyl, each of which may be unsubstituted or further substituted with R⁶, or any C₁-C₆ alkyl may also be substituted with C₃-C₈ cycloalkyl or with phenoxy which may be unsubstituted or substituted with R⁶ or with phenyl, furyl, thienyl, pyrrolyl, each of which may be unsubstituted or substituted with R⁶, or
R⁵ is H, halo or C₁₋₆ alkyl optionally substituted with oxo;
R⁶ is halo, CF₃, C₁₋₆ alkyl optionally substituted with oxo or hydroxy, or C₁₋₆ alkoxy optionally substituted with fluoro;
wherein R³ may be attached to the heterocyclic moiety of the compound of Formula I at either the 4 or 5 position, and, accordingly, the remaining portion of the molecule will be attached at the remaining available carbon atom; or
a pharmaceutically acceptable salt, stereoisomer, enantiomer, racemate or combination thereof.

13. The method of claim 12, wherein compound is selected from the group consisting of

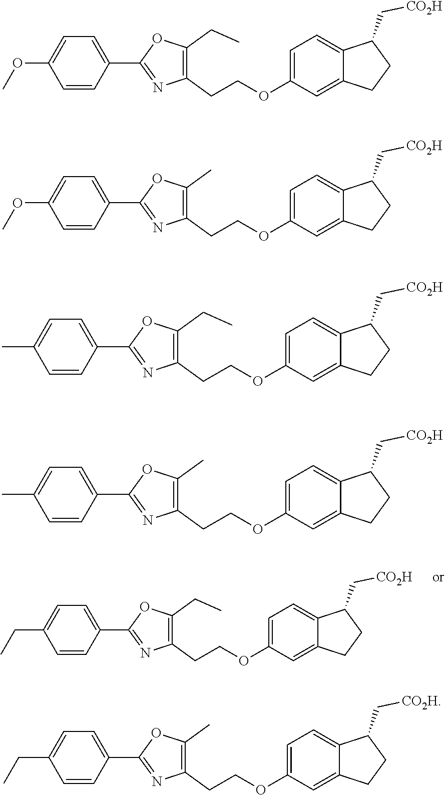 [SMILES: CCc1oc(-c2ccc(OC)cc2)nc1CCOc1ccc2c(c1)CC[C@H]2CC(=O)O ; COc1ccc(-c2nc(CCOc3ccc4c(c3)CC[C@H]4CC(=O)O)c(C)o2)cc1 ; CCc1oc(-c2ccc(C)cc2)nc1CCOc1ccc2c(c1)CC[C@H]2CC(=O)O ; Cc1ccc(-c2nc(CCOc3ccc4c(c3)CC[C@H]4CC(=O)O)c(C)o2)cc1 ; CCc1ccc(-c2nc(CCOc3ccc4c(c3)CC[C@H]4CC(=O)O)c(CC)o2)cc1 ; CCc1ccc(-c2nc(CCOc3ccc4c(c3)CC[C@H]4CC(=O)O)c(C)o2)cc1]

14. A method of claim 12 comprising administrating a therapeutically effective amount of: 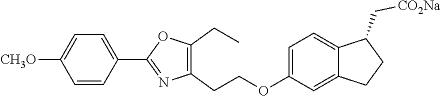
or
a pharmaceutically acceptable salt thereof.

15. A method of treating cerebral amyloid angiopathy (CAA) comprising administrating a therapeutically effective amount of a compound of Formula I: 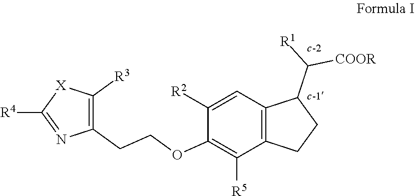
wherein
R is H, Na⁺, Li⁺, Ca⁺, K⁺, N⁺(C₁₋₆)₄, or C₁₋₆ alkyl;
R¹ is H, C₁₋₆ alkyl, C₃₋₆ cycloalkyl, or C₂₋₆ alkenyl, or C₁₋₆ alkoxy, each of which may be unsubstituted or substituted with fluoro, or phenyl which may be unsubstituted or substituted with R⁶, “c-2” is defined as the second carbon of the acetic acid portion of Formula I, and “c-1′” is the first carbon of the indane group of Formula I;
R² is H, halo, or C₁₋₆ alkyl which may be unsubstituted or substituted with C₁₋₆ alkoxy, oxo, fluoro, or
R² is phenyl, furyl, thienyl, pyrrolyl, oxazolyl, thiazolyl, imidazolyl, pyrazolyl, pyridyl, pyrrolidinyl, piperidinyl, tetrahydropyranyl, piperazinyl, or morpholinyl, each of which may be unsubstituted or substituted with R⁶;
R³ is H, C₁₋₆ alkyl, or phenyl, which may be unsubstituted or substituted with R⁶;
X is O or S;
R⁴ is phenyl, naphthyl, furyl, thienyl, pyrrolyl, oxazolyl, thiazolyl, pyrazolyl, pyridyl, pyrimidinyl, benzofuryl, benzothienyl, indolyl, indazolyl, benzoxazolyl, benzimidazolyl, quinolyl, isoquinolyl, or 1,4-benzodioxanyl, each of which may be unsubstituted or singlularly or multiply substituted with R⁶, or with phenyl, furyl, thienyl, pyrrolyl, each of which may be unsubstituted or singularly or multiply substituted with R⁶; or
R⁴ is C₁-C₆ alkyl or C₃-C₈ cycloalkyl, either of which may be unsubstituted or substituted with fluoro, oxo, or C₁-C₆ alkoxy which may be unsubstituted or substituted with C₁-C₆ alkoxy, or phenyl optionally substituted with R⁶, each of which may be substituted with phenyl, naphthyl, furyl, thienyl, pyrrolyl, each of which may be unsubstituted or further substituted with R⁶, or any C₁-C₆ alkyl may also be substituted with C₃-C₈ cycloalkyl or with phenoxy which may be unsubstituted or substituted with R⁶ or with phenyl, furyl, thienyl, pyrrolyl, each of which may be unsubstituted or substituted with R⁶, or
R⁵ is H, halo or C₁₋₆ alkyl optionally substituted with oxo;
R⁶ is halo, CF₃, C₁₋₆ alkyl optionally substituted with oxo or hydroxy, or C₁₋₆ alkoxy optionally substituted with fluoro;
wherein R³ may be attached to the heterocyclic moiety of the compound of Formula I at either the 4 or 5 position, and, accordingly, the remaining portion of the molecule will be attached at the remaining available carbon atom; or
a pharmaceutically acceptable salt, stereoisomer, enantiomer, racemate or combination thereof.

16. The method of claim 15, wherein compound is selected from the group consisting of

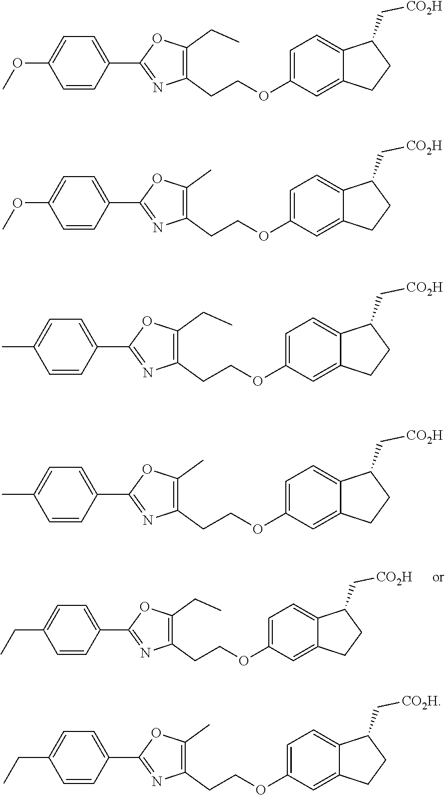 [SMILES: CCc1oc(-c2ccc(OC)cc2)nc1CCOc1ccc2c(c1)CC[C@H]2CC(=O)O ; COc1ccc(-c2nc(CCOc3ccc4c(c3)CC[C@H]4CC(=O)O)c(C)o2)cc1 ; CCc1oc(-c2ccc(C)cc2)nc1CCOc1ccc2c(c1)CC[C@H]2CC(=O)O ; Cc1ccc(-c2nc(CCOc3ccc4c(c3)CC[C@H]4CC(=O)O)c(C)o2)cc1 ; CCc1ccc(-c2nc(CCOc3ccc4c(c3)CC[C@H]4CC(=O)O)c(CC)o2)cc1 ; CCc1ccc(-c2nc(CCOc3ccc4c(c3)CC[C@H]4CC(=O)O)c(C)o2)cc1]

17. The method of of claim 15 comprising administrating a therapeutically effective amount of: 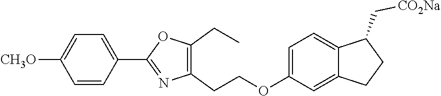
or
a pharmaceutically acceptable salt thereof.
